(19) United States
(12) Patent Application Publication
(10) Pub. No.: US 2011/0212923 A1
(43) Pub. Date: Sep. 1, 2011

(54) Phosphate Derivatives of Substituted Benzoxazoles

(75) Inventors: Reinhardt Baudy, Lower Saucon, PA (US); Eugene J. Trybulski, Huntington Valley, PA (US); Ronald Magolda, Wallingford, PA (US); Diane Schneider, Wallingford, PA (US); Gulnaz Khafizova, West Nyack, NY (US); Mahdi B. Fawzi, Morristown, NJ (US); Cuijian Yang, Collegeville, PA (US)

(73) Assignee: PFIZER INC.

(21) Appl. No.: 12/866,477

(22) PCT Filed: Feb. 6, 2009
(86) PCT No.: PCT/US2009/033398

§ 371 Date: Apr. 26, 2011

Related U.S. Application Data
(60) Provisional application No. 61/027,133, filed on Feb. 8, 2008.

(51) Int. Cl. A61K 31/675; C07F 9/653; C07D 263/57; A61K 31/423; A61P 29/00; A61P 35/00; A61P 9/00; A61P 25/00; A61P 17/00; A61P 3/10; A61P 19/02; A61P 11/06; A61P 3/00; A61P 3/06; A61P 9/10; A61P 9/12; A61P 25/28; A61P 25/22; A61P 17/06; A61P 17/04; A61P 15/00; A61P 13/00

(52) U.S. Cl. 514/80; 548/113; 548/224; 514/375

(57) ABSTRACT

The present invention relates to phosphate derivatives of estrogen receptor beta agonists, compositions thereof, preparations thereof, and uses thereof. Formula (I).

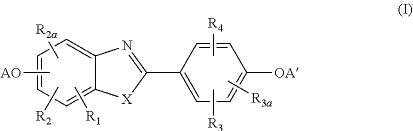

### FIELD OF THE INVENTION

This invention relates to phosphate derivatives of substituted benzoxazoles and salts thereof, to compositions comprising the same, and to methods of making and using the same.

### BACKGROUND OF THE INVENTION

Compounds having estrogenic activity are disclosed in U.S. Pat. No. 6,794,403, which is hereby incorporated by reference in its entirety. Given the importance of these compounds, it can be seen that a continuing need exists for developing new compounds with improved properties and new processes for preparation of the same.

Compounds, such as those disclosed in U.S. Pat. No. 6,794,403, have been shown to have therapeutic activity in the treatment and inhibition of several diseases and disorders. There exists a need for new compounds which exhibit, inter alia, an improved solubility profile. This invention is directed to this, as well as other, important ends.

### SUMMARY OF THE INVENTION

In some embodiments, the present invention provides compounds of Formula I, having the structure:

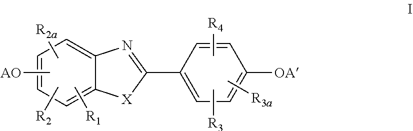

wherein:

R₁ is hydrogen, hydroxyl, halogen, alkyl of 1-6 carbon atoms, trifluoroalkyl of 1-6 carbon atoms, cycloalkyl of 3-8 carbon atoms, alkoxy of 1-6 carbon atoms, trifluoroalkoxy of 1-6 carbon atoms, thioalkyl of 1-6 carbon atoms, sulfoxoalkyl of 1-6 carbon atoms, sulfonoalkyl of 1-6 carbon atoms, aryl of 6-10 carbon atoms, a 5 or 6-membered heterocyclic ring having 1 to 4 heteroatoms selected from O, N, S, —NO₂, NR₅R₆, —N(R₅)COR₆, —CN, —CHFCN, CF₂CN, alkynyl of 2-7 carbon atoms, alkenyl of 2-7 carbon atoms; wherein the alkyl or alkenyl moieties are optionally substituted with hydroxyl, —CN, halogen, trifluoroalkyl, trifluoroalkoxy, —COR₅, —CO₂R₅, —NO₂, CONR₅R₆, NR₅R₆, or N(R₅)COR₆;

R₂ and R₂ₐ are each, independently, hydrogen, hydroxyl, halogen, alkyl of 1-6 carbon atoms, alkoxy of 1-4 carbon atoms, alkenyl of 2-7 carbon atoms, alkynyl of 2-7 carbon atoms, trifluoralkyl of 1-6 carbon atoms, or trifluoroalkoxy of 1-6 carbon atoms; wherein the alkyl, alkenyl, or alkynyl moieties are optionally substituted with hydroxyl, —CN, halogen, trifluoroalkyl, trifluoroalkoxy, —COR₅, —CO₂R₅, —NO₂, CONR₅R₆, NR₅R₆, or N(R₅)COR₆;

R₃ and R₃ₐ are each, independently, hydrogen, alkyl of 1-6 carbon atoms, alkenyl of 2-7 carbon atoms, alkynyl of 2-7 carbon atoms, halogen, alkoxy of 1-4 carbon atoms, trifluoroalkyl of 1-6 carbon atoms, or trifluoroalkoxy of 1-6 carbon atoms; wherein the alkyl, alkenyl, or alkynyl moieties are optionally substituted with hydroxyl, —CN, halogen, trifluoroalkyl, trifluoroalkoxy, —COR₅, —CO₂R₅, —NO₂, CONR₅R₆, NR₅R₆ or N(R₅)COR₆;

R₄ is hydrogen, halogen, or alkyl of 1-6 carbon atoms; provided that when R₄ is hydrogen, R₁, R₂, R₂ₐ, R₃, and R₃ₐ, cannot all be hydrogen.

R₅ and R₆ are each, independently, hydrogen, alkyl of 1-6 carbon atoms, aryl of 6-10 carbon atoms;

X is O, S, or NR₇; and

R₇ is hydrogen, alkyl of 1-6 carbon atoms, aryl of 6-10 carbon atoms, —COR₅, —CO₂R₅ or SO₂R₅;

A and A′ are each independently hydrogen, a protecting group, or —P(O)(OR₈)(OR₉); wherein at least one of A or A′ is —P(O)(OR₈)(OR₉);

R₈ and R₉ are each independently selected from H, a protecting group, C₁₋₁₀ alkyl, C₁₋₁₀ haloalkyl, C₂₋₁₀ alkenyl, C₂₋₁₀ alkynyl, aryl, cycloalkyl, heteroaryl, heterocycloalkyl, arylalkyl, heteroarylalkyl, cycloalkylalkyl and heterocycloalkylalkyl, wherein each of the C₁₋₁₀ alkyl, C₁₋₁₀ haloalkyl, C₂₋₁₀ alkenyl, C₂₋₁₀ alkynyl, aryl, cycloalkyl, heteroaryl, heterocycloalkyl, arylalkyl, heteroarylalkyl, cycloalkylalkyl and heterocycloalkylalky is optionally substituted by 1, 2, 3, 4 or 5 R₁₀;

each R₁₀ is independently halo, C₁₋₆ alkyl, C₁₋₆ haloalkyl, aryl, cycloalkyl, heteroaryl, heterocycloalkyl, CN, NO₂, OR^{a}, SR^{a}, C(═O)R^{b}, C(═O)NR^{c}R^{d}, C(═O)OR^{a}, OC(═O)R^{b}, OC(═O)NR^{c}R^{d}, NR^{c}R^{d}, NR^{c}C(═O)R^{b}, NR^{c}C(═O)OR^{a}, NR^{c}S(═O)₂R^{b}, S(═O)R^{b}, S(═O)NR^{c}R^{d}, S(═O)₂R^{b}, or S(═O)₂NR^{c}R^{d};

each R^{a} is independently selected from H, C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl and heterocycloalkylalkyl, wherein each of said C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl and heterocycloalkylalkyl is optionally substituted by OH, C₁₋₆ alkoxy, C₁₋₆ haloalkoxy, amino, halo, C₁₋₆ alkyl, C₁₋₆ haloalkyl, aryl, arylalkyl, heteroaryl, heteroarylalkyl, cycloalkyl or heterocycloalkyl;

each R^{b} is independently selected from H, C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl and heterocycloalkylalkyl, wherein each of said C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl and heterocycloalkylalkyl is optionally substituted by OH, C₁₋₆ alkoxy, C₁₋₆ haloalkoxy, amino, halo, C₁₋₆ alkyl, C₁₋₆ haloalkyl, aryl, arylalkyl, heteroaryl, heteroarylalkyl, cycloalkyl or heterocycloalkyl; and

R^{c} and R^{d} are each, independently, selected from H, C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl and heterocycloalkylalkyl, wherein each of said C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl and heterocycloalkylalkyl is optionally substituted by OH, C₁₋₆ alkoxy, C₁₋₆ haloalkoxy, amino, halo, C₁₋₆ alkyl, C₁₋₆ haloalkyl, aryl, arylalkyl, heteroaryl, heteroarylalkyl, cycloalkyl or heterocycloalkyl; or

R^{c} and R^{d} together with the N atom to which they are attached form a 4-, 5-, 6- or 7-membered heterocycloalkyl group;

or a pharmaceutically acceptable salt thereof.

In some embodiments, the present invention provides compounds having the structure of Formula Ia:

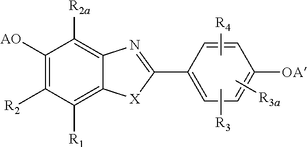

### Ia

or a pharmaceutically acceptable salt thereof.

Some embodiments of the present invention provide a compound having the structure of Formula II:

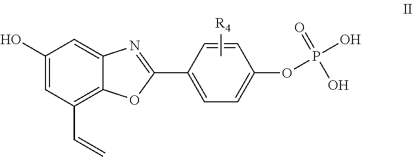

or a pharmaceutically acceptable salt thereof, wherein R₄ is as set forth above for Formula I.

In some embodiments, the present invention provides methods for treating or inhibiting a disease, disorder, or condition in a mammal, comprising the steps of: a) identifying a mammal having said disease, disorder, or condition; and b) administering to said mammal a therapeutically effective amount of a compound of Formula I or Ia; and wherein said disease, disorder, or condition is selected from: an inflammatory disease, disorder, or condition; cancer; a cardiovascular disease, disorder, or condition; a cognitive disease, disorder, or condition; a disease, disorder, or condition of the skin; a neurodegenerative disease, disorder, or condition; diabetes; a disease, disorder, or condition associated with peri-menopause, menopause, or post-menopause; and a disease, disorder or condition associated with a dysregulated systemic inflammatory response.

In some embodiments, the present invention provides processes for preparing compounds of Formula I as described above, comprising the step of: phosphorylating a compound of Formula IV:

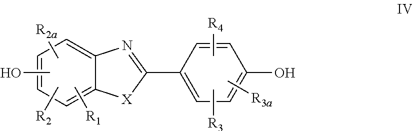

or a salt thereof with a phosphorylating reagent; wherein R₁, R₂, R₂ₐ, R₃, R₃ₐ, R₄, and X are as set forth above for Formula I.

### DESCRIPTION OF THE INVENTION

Aspects of the present invention provide phosphate derivatives of substituted benzoxazoles and salts thereof, compositions comprising the same, methods of making such phosphate derivatives of substituted benzoxazoles, salts and compositions, and methods of using the substituted benzoxazoles, salts and compositions.

The phosphate derivatives of substituted benzoxazoles may be useful as prodrugs of the substituted benzoxazoles, wherein the prodrug provides different properties relative to the substituted benzoxazole active agent but which prodrug is metabolized to the substituted benzoxazole active agent in an individual to whom the prodrug is administered. In some embodiments, the prodrug may differ from the active agent with respect to one or more following properties: solubility in aqueous solutions, particularly such solutions suitable for injectable administration; stability in solution and/or in crystal and/or uncrystalized form; ease and/or efficiency of manufacture (synthesis and/or purification) and/or handling; ease of use; and/or in vivo activity including, but not limited to toxicity, bioavailability, and/or half-life of the prodrug or active metabolite thereof.

The addition of the phosphate group or groups to the substituted benzoxazoles can result in three forms: two mono-phosphate forms and a di-phosphate form. In some embodiments, synthesis and/or purification provides one of the three forms as a predominant product. In some embodiments, synthesis of the phosphate derivatives of substituted benzoxazoles provides selective addition of the phosphate group to one site to produce a one of the two monohydrate forms as a predominant product. In some embodiments, synthesis and/or purification provides high yields of the phosphate derivatives of substituted benzoxazoles. In some embodiments, synthesis and/or purification of the phosphate derivatives of substituted benzoxazoles can be performed under conditions which minimize production of contaminants.

In some embodiments, the phosphate derivatives of substituted benzoxazoles are useful in the treatment or inhibition of a disease, disorder, or condition in a mammal selected from: an inflammatory disease, disorder, or condition; cancer; a cardiovascular disease, disorder, or condition; a cognitive disease, disorder, or condition; a disease, disorder, or condition of the skin; a neurodegenerative disease, disorder, or condition; diabetes; a disease, disorder, or condition associated with peri-menopause, menopause, or post-menopause; benign or malignant abnormal tissue growth including; and a disease, disorder or condition associated with a dysregulated systemic inflammatory response. In some embodiments, the treatment or inhibition of such diseases and disorders is preferably achieved by parental administration. Phosphate derivatives of substituted benzoxazoles which have increased solubility in aqueous solutions, particularly such solutions suitable for injectable administration, relative to substituted benzoxazoles allow for the delivery of the therapeutically effective substituted benzoxazoles in an injectable form by providing for a soluble derivative of the substituted benzoxazoles which is converted to the therapeutically effective substituted benzoxazoles when metabolized by the individual.

The treatment and inhibition of sepsis is one such disease or disorder in which parental administration of therapeutic agents may be desirable compared to other routes of administration. Sepsis is an amplified and dysregulated systemic inflammatory response (SIRS) to infection which remains a profound outcome in even previously normal patients. SIRS is defined as at least 2 of the 4 clinical signs: hypo- or hyperthermia, tachycardia, tachypnea or hyperventilation, or abnormal leukogram. Sepsis is defined as SIRS plus documented or suspected infection, and the addition of one organ dysfunction/failure is severe sepsis. Severe sepsis with hypoperfusion and/or intractable hypotension is septic shock, and severe sepsis with more than one organ failure is multiple organ failure (MOF). Despite 25 years of active research exploring multiple therapeutic avenues for sepsis, only one novel agent (Xigris, recombinant human activated protein C) which has demonstrated only marginal efficacy, has been approved for treatment of severe sepsis in patients with a high risk of death.

Severe Sepsis occurs typically in response to severe documented or suspected infection. Despite advances in critical care medicine, new antibiotics, and the approval of Xigris, recombinant activated Protein C (rAPC), the syndrome remains a large unmet medical need as it continues to exhibit about 30% mortality, ranging from 20-80% mortality, increasing in severity with patient age. A safe, well-tolerated therapeutic agent that could treat severe sepsis and stop the progression to severe sepsis with multiple organ failure (MOF) or septic shock (SS), thereby improving survival rates, would clearly provide an innovative solution.

In some embodiments, the present invention provides compounds of Formula I, having the structure:

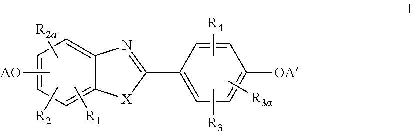

wherein:

R₁ is hydrogen, hydroxyl, halogen, alkyl of 1-6 carbon atoms, trifluoroalkyl of 1-6 carbon atoms, cycloalkyl of 3-8 carbon atoms, alkoxy of 1-6 carbon atoms, trifluoroalkoxy of 1-6 carbon atoms, thioalkyl of 1-6 carbon atoms, sulfoxoalkyl of 1-6 carbon atoms, sulfonoalkyl of 1-6 carbon atoms, aryl of 6-10 carbon atoms, a 5 or 6-membered heterocyclic ring having 1 to 4 heteroatoms selected from O, N, S, —NO₂, NR₅R₆, —N(R₅)COR₆, —CN, —CHFCN, CF₂CN, alkynyl of 2-7 carbon atoms, alkenyl of 2-7 carbon atoms; wherein the alkyl or alkenyl moieties are optionally substituted with hydroxyl, —CN, halogen, trifluoroalkyl, trifluoroalkoxy, —COR₅, —CO₂R₅, —NO₂, CONR₅R₆, NR₅R₆, or N(R₅)COR₆;

R₂ and R₂ₐ are each, independently, hydrogen, hydroxyl, halogen, alkyl of 1-6 carbon atoms, alkoxy of 1-4 carbon atoms, alkenyl of 2-7 carbon atoms, alkynyl of 2-7 carbon atoms, trifluoralkyl of 1-6 carbon atoms, or trifluoroalkoxy of 1-6 carbon atoms; wherein the alkyl, alkenyl, or alkynyl moieties are optionally substituted with hydroxyl, —CN, halogen, trifluoroalkyl, trifluoroalkoxy, —COR₅, —CO₂R₅, —NO₂, CONR₅R₆, NR₅R₆, or N(R₅)COR₆;

R₃ and R₃ₐ are each, independently, hydrogen, alkyl of 1-6 carbon atoms, alkenyl of 2-7 carbon atoms, alkynyl of 2-7 carbon atoms, halogen, alkoxy of 1-4 carbon atoms, trifluoroalkyl of 1-6 carbon atoms, or trifluoroalkoxy of 1-6 carbon atoms; wherein the alkyl, alkenyl, or alkynyl moieties are optionally substituted with hydroxyl, —CN, halogen, trifluoroalkyl, trifluoroalkoxy, —COR₅, —CO₂R₅, —NO₂, CONR₅R₆, NR₅R₆ or N(R₅)COR₆;

R₄ is hydrogen, halogen, or alkyl of 1-6 carbon atoms; with the proviso that when R₄ is hydrogen, R₁, R₂, R₂ₐ, R₃, and R₃ₐ, cannot all be hydrogen.

R₅ and R₆ are each, independently, hydrogen, alkyl of 1-6 carbon atoms, aryl of 6-10 carbon atoms;

X is O, S, or NR₇; and

R₇ is hydrogen, alkyl of 1-6 carbon atoms, aryl of 6-10 carbon atoms, —COR₅, —CO₂R₅ or SO₂R₅;

A and A′ are each independently hydrogen, a protecting group, or —P(O)(OR₈)(OR₉); wherein at least one of A or A′ is —P(O)(OR₈)(OR₉);

R₈ and R₉ are each independently selected from H, a protecting group, C₁₋₁₀ alkyl, C₁₋₁₀ haloalkyl, C₂₋₁₀ alkenyl, C₂₋₁₀ alkynyl, aryl, cycloalkyl, heteroaryl, heterocycloalkyl, arylalkyl, heteroarylalkyl, cycloalkylalkyl and heterocycloalkylalkyl, wherein each of the C₁₋₁₀ alkyl, C₁₋₁₀ haloalkyl, C₂₋₁₀ alkenyl, C₂₋₁₀ alkynyl, aryl, cycloalkyl, heteroaryl, heterocycloalkyl, arylalkyl, heteroarylalkyl, cycloalkylalkyl and heterocycloalkylalky is optionally substituted by 1, 2, 3, 4 or 5 R₁₀;

each R₁₀ is independently halo, C₁₋₆ alkyl, C₁₋₆ haloalkyl, aryl, cycloalkyl, heteroaryl, heterocycloalkyl, CN, NO₂, OR^{a}, SR^{a}, C(═O)R^{b}, C(═O)NR^{c}R^{d}, C(═O)OR^{a}, OC(═O)R^{b}, OC(═O)NR^{c}R^{d}, NR^{c}R^{d}, NR^{c}C(═O)R^{b}, NR^{c}C(═O)OR^{a}, NR^{c}S(═O)₂R^{b}, S(═O)R^{b}, S(═O)NR^{c}R^{d}, S(═O)₂R^{b}, or S(═O)₂NR^{c}R^{d};

each R^{a} is independently selected from H, C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl and heterocycloalkylalkyl, wherein each of said C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl and heterocycloalkylalkyl is optionally substituted by OH, C₁₋₆ alkoxy, C₁₋₆ haloalkoxy, amino, halo, C₁₋₆ alkyl, C₁₋₆ haloalkyl, aryl, arylalkyl, heteroaryl, heteroarylalkyl, cycloalkyl or heterocycloalkyl;

each R^{b} is independently selected from H, C₁₋₆ alkyl, C₁₋₆, haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl and heterocycloalkylalkyl, wherein each of said C₁₋₆ alkyl, C₁₋₆ haloalkyl, alkenyl, C₂₋₆ alkynyl, aryl, cycloalkyl, heteroaryl, heterocycloalkyl, arylalkyl, heteroarylalkyl, cycloalkylalkyl and heterocycloalkylalkyl is optionally substituted by OH, C₁₋₆ alkoxy, C₁₋₆ haloalkoxy, amino, halo, C₁₋₆ alkyl, C₁₋₆ haloalkyl, aryl, arylalkyl, heteroaryl, heteroarylalkyl, cycloalkyl or heterocycloalkyl; and

R^{c} and R^{d} are each, independently, selected from H, C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl and heterocycloalkylalkyl, wherein each of said C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl and heterocycloalkylalkyl is optionally substituted by OH, C₁₋₆ alkoxy, C₁₋₆ haloalkoxy, amino, halo, C₁₋₆ alkyl, C₁₋₆ haloalkyl, aryl, arylalkyl, heteroaryl, heteroarylalkyl, cycloalkyl or heterocycloalkyl; or

R^{c} and R^{d} together with the N atom to which they are attached form a 4-, 5-, 6- or 7-membered heterocycloalkyl group;

or a pharmaceutically acceptable salt thereof.

In some embodiments, R₈ and R₉ are each independently selected from H, a protecting group, C₁₋₁₀ alkyl, C₁₋₁₀ haloalkyl, C₂₋₁₀ alkenyl, C₂₋₁₀ alkynyl, aryl, cycloalkyl, heteroaryl, heterocycloalkyl, arylalkyl, heteroarylalkyl, cycloalkylalkyl or heterocycloalkylalkyl, wherein each of the C₁₋₁₀ alkyl, C₁₋₁₀ haloalkyl, C₂₋₁₀ alkenyl, C₂₋₁₀ alkynyl, aryl, cycloalkyl, heteroaryl, heterocycloalkyl, arylalkyl, heteroarylalkyl, cycloalkylalkyl and heterocycloalkylalky is optionally substituted by 1, 2, 3, 4 or 5 substituents independently selected from halo, C₁₋₄ alkyl, C₁₋₄ haloalkyl, aryl, cycloalkyl, heteroaryl, heterocycloalkyl, heterocycloalkyl, arylalkyl, heteroarylalkyl, cycloalkylalkyl, heterocycloalkylalkyl, CN, NO₂, OH, C₁₋₄ alkoxy, C₁₋₄ haloalkoxy, aryloxy, heteroaryloxy, arylalkyloxy, heteroarylalkyloxy, amino, C₁₋₄ alkylamino, C₂₋₈ dialkylamino, SH, —S—(C₁₋₄ alkyl), C(═O)H, C(═O)—(C₁₋₄ alkyl), C(═O)-(aryl), C(═O)-(arylalkyl), C(═O)NH₂, C(═O)NH(C₁₋₄ alkyl), C(═O)N(C₁₋₄ alkyl)₂, C(═O)OH, C(═O)O—(C₁₋₄ alkyl), C(═O)O-(arylalkyl), OC(═O)H, OC(═O)—(C₁₋₄ alkyl), OC(═O)-(aryl), OC(═O)-(arylalkyl), OC(═O)NH₂, OC(═O)NH(C₁₋₄ alkyl), OC(═O)NH-(arylalkyl), OC(═O)N(C₁₋₄ alkyl)₂, NHC(═O)—(C₁₋₄ alkyl), NHC(═O)-(aryl), NHC(═O)-(arylalkyl), N(C₁₋₄ alkyl)C(═O)—(C₁₋₄ alkyl), N(C₁₋₄ alkyl)C(═O)-(aryl), N(C₁₋₄ alkyl)C(═O)-(arylalkyl), NHC(═O)O-(arylalkyl), NHC(═O)O—(C₁₋₄ alkyl), NHC(═O)O-(arylalkyl), NHC(═O)NH(C₁₋₄ alkyl), NHC(═O)NH-(aryl), NHC(═O)NH-(arylalkyl), NHC(═O)NH(C₁₋₄ alkyl)₂, N(C₁₋₄ alkyl)C(═O)NH(C₁₋₄ alkyl), N(C₁₋₄ alkyl)C(═O)NH-(aryl), N(C₁₋₄ alkyl)C(═O)NH-(arylalkyl), N(C₁₋₄ alkyl)C(═O)NH(C₁₋₄ alkyl)₂, NHS(═O)₂—(C₁₋₄ alkyl), NHS(═O)₂-(aryl), NHS(═O)₂-(arylalkyl), S(═O)₂—(C₁₋₄ alkyl), S(═O)₂-(aryl), S(═O)₂-(arylalkyl), S(═O)₂NH(C₁₋₄ alkyl), S(═O)₂NH(aryl), and S(═O)₂NH(arylalkyl).

In some further embodiments, R₈ and R₉ are each independently selected from H, a protecting group, C₁₋₁₀ alkyl, C₁₋₁₀ haloalkyl, C₂₋₁₉ alkenyl, C₂₋₁₀ alkynyl, aryl, cycloalkyl, heteroaryl, heterocycloalkyl, arylalkyl, heteroarylalkyl, cycloalkylalkyl and heterocycloalkylalkyl, wherein each of the C₁₋₁₀ alkyl, C₁₋₁₀ haloalkyl, C₂₋₁₀ alkenyl, C₂₋₁₀ alkynyl, aryl, cycloalkyl, heteroaryl, heterocycloalkyl, arylalkyl, heteroarylalkyl, cycloalkylalkyl and heterocycloalkylalkyl is optionally substituted by 1, 2 or 3 substituents independently selected from halo, C₁₋₄ alkyl, C₁₋₄ haloalkyl, aryl, cycloalkyl, heteroaryl, heterocycloalkyl, heterocycloalkyl, arylalkyl, heteroarylalkyl, cycloalkylalkyl, heterocycloalkylalkyl, CN, NO₂, OH, C₁₋₄ alkoxy, C₁₋₄ haloalkoxy, amino, C₁₋₄ alkylamino and C₂₋₈ dialkylamino.

In yet other embodiments, R₈ and R₉ are each independently selected from H, a protecting group, C₁₋₁₀ alkyl, C₁₋₁₀ haloalkyl, C₂₋₁₀ alkenyl, C₂₋₁₀ alkynyl, aryl, cycloalkyl, heteroaryl, heterocycloalkyl, arylalkyl, heteroarylalkyl, cycloalkylalkyl and heterocycloalkylalkyl. In some embodiments, R₈ and R₉ are each independently selected from H, a protecting group, C₁₋₁₀ alkyl and C₁₋₁₀ haloalkyl. In some embodiments, R₈ and R₉ are each independently selected from H, a protecting group, C₁₋₆ alkyl and C₁₋₆ haloalkyl. In some further embodiments, R₈ and R₉ are each independently selected from H, a protecting group and C₁₋₆ alkyl. In yet further embodiments, R₈ and R₉ are each independently selected from H, a protecting group, and C₁₋₄ alkyl.

In some embodiments, the present invention provides compounds wherein R₁ is alkenyl of 2-7 carbon atoms; wherein the alkenyl moiety is optionally substituted with hydroxyl, —CN, halogen, trifluoroalkyl, trifluoroalkoxy, —COR₅, —CO₂R₅, —NO₂, CONR₅R₆, NR₅R₆, or N(R₅)COR₆.

In some embodiments, the present invention provides phosphate derivatives of a compound selected from the group consisting of: 2-(3-fluoro-4-hydroxyphenyl)-1,3-benzoxazol-5-ol, 2-(3-chloro-4-hydroxyphenyl)-1,3-benzoxazol-5-ol, 2-(3-fluoro-4-hydroxyphenyl)-7-vinyl-1,3-benzoxazol-5-ol, 2-(2-chloro-4-hydroxyphenyl)-1,3-benzoxazol-5-ol, 2-(3-fluoro-4-hydroxyphenyl)-1,3-benzoxazol-6-ol, 2-(3-tert-butyl-4-hydroxyphenyl)-1,3-benzoxazol-6-ol, 2-(3-chloro-4-hydroxyphenyl)-1,3-benzoxazol-6-ol, 6-chloro-2-(3-fluoro-4-hydroxyphenyl)-1,3-benzoxazol-5-ol, 6-bromo-2-(3-fluoro-4-hydroxyphenyl)-1,3-benzoxazol-5-ol, 6-chloro-2-(4-hydroxyphenyl)-1,3-benzoxazol-5-ol, 5 -chloro-2-(4-hydroxyphenyl)-1,3-benzoxazol-6-ol, 7-bromo-2-(3-fluoro-4-hydroxyphenyl)-1,3-benzoxazol-5-ol, 7-bromo-2-(2-fluoro-4-hydroxyphenyl)-1,3-benzoxazol-5-ol, 7-bromo-2-(2,3-difluoro-4-hydroxyphenyl)- 1,3-benzoxazol-5-ol, 2-(4-hydroxyphenyl)-7-vinyl-1,3-benzoxazol-5-ol, 7-(1,2-dibromoethyl)-2-(4-hydroxyphenyl)-1,3-benzoxazol-5-ol, 7-(1-bromovinyl)-2-(4-hydroxyphenyl)-1,3-benzoxazol-5-ol, 7-ethynyl-2-(4-hydroxyphenyl)-1,3-benzoxazol-5-ol, 2-(4-hydroxyphenyl)-7-propyl-1,3-benzoxazol-5-ol, 7-butyl-2-(4-hydroxyphenyl)-1,3-benzoxazol-5-ol, 7-cyclopentyl-2-(4-hydroxyphenyl)-1,3-benzoxazol-5-ol, ethyl 5-hydroxy-2-(4-hydroxyphenyl)-1,3-benzoxazole-7-carboxylate, 2-(4-hydroxyphenyl)-7-phenyl-1,3-benzoxazol-5-ol, 2-(4-hydroxyphenyl)-7-methoxy-1,3-benzoxazol-5-ol, 7-ethyl-2-(4-hydroxyphenyl)-1,3-benzoxazol-5-ol, 7-ethyl-2-(2-ethyl-4-hydroxyphenyl)-1,3-benzoxazol-5-ol, 5-hydroxy-2-(4-hydroxyphenyl)-1,3-benzoxazole-7-carbaldehyde, 7-(hydroxymethyl)-2-(4-hydroxyphenyl)-1,3-benzoxazol-5-ol, 7-(bromomethyl)-2-(4-hydroxyphenyl)-1,3-benzoxazol-5-ol, [5-hydroxy-2-(4-hydroxyphenyl)-1,3-benzoxazol-7-yl] acetonitrile, 7-(1-hydroxy-1-methylethyl)-2-(4-hydroxyphenyl)-1,3-benzoxazol-5-ol, 2-(4-hydroxyphenyl)-7-isopropenyl-1,3-benzoxazol-5-ol, 2-(4-hydroxyphenyl)-7-isopropyl-1,3-benzoxazol-5-ol, 7-bromo-2-(4-hydroxy-3-(trifluoromethyl)phenyl)-1,3-benzoxazol-5-ol, 7-(2-furyl)-2-(4-hydroxyphenyl)-1,3-benzoxazol-5-ol, 2-(3-fluoro-4-hydroxyphenyl)-7-(2-furyl)-1,3-benzoxazol-5-ol, 2-(4-hydroxyphenyl)-7-thien-2-yl-1,3-benzoxazol-5-ol, 2-(4-hydroxyphenyl)-7-(1,3-thiazol-2-yl)-1,3-benzoxazol-5-ol, 2-(3-fluoro-4-hydroxyphenyl)-5-hydroxy-1,3-benzoxazole-7-carbonitrile, 4-bromo-2-(4-hydroxyphenyl)-7-methoxy-1,3-benzoxazol-5-ol, 4,6-dibromo-2-(4-hydroxyphenyl)-7-methoxy-1,3-benzoxazol-5-ol, and 7-bromo-2-(3,5-difluoro-4-hydroxyphenyl)-1,3-benzoxazol-5-ol; or a pharmaceutically acceptable salt thereof.

As used herein, the term “phosphate derivative” is meant to include monophosphate derivatives of the compounds described herein and diphosphate (also referred to as bisphosphate) derivatives of the compounds described herein. For example, each compound in the above list can be phosphorylated at either one of its hydroxyl substituents or at both of its hydroxyl substituents. That is, some embodiments of compounds having Formula I are monophosphates in which A is —P(O)(OR₈)(OR₉), preferably P(O)(OH)(OH), and A′ is hydrogen or a protecting group, preferably hydrogen; in some preferred embodiments of such monophosphates, A is P(O)(OH)(OH) and A′ is hydrogen. Some embodiments of compounds having Formula I are monophosphates in which A is hydrogen or a protecting group, preferably hydrogen, and A′ is —P(O)(OR₈)(OR₉), preferably P(O)(OH)(OH); in some preferred embodiments of such monophosphates A is hydrogen and A′ is P(O)(OH)(OH). Some embodiments of compounds having Formula I are diphophosphates (also referred to as bisphosphates) in which both A and A′ are —P(O)(OR₈)(OR₉); in some embodiments, at least one of A and A′ is P(O)(OH)(OH); in some embodiments both A and A′ are P(O)(OH)(OH).

In some embodiments, the present invention provides compounds having the structure of Formula Ia:

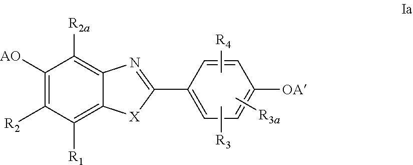

or a pharmaceutically acceptable salt thereof.

In some embodiments, A′ is —P(O)(OR⁸)(OR⁹); X is O; R₁ is ethylene; R₂, R₂ₐ, R₃, and R₃ₐ are hydrogen; and R₄ is halogen. In some embodiments, R₄ is fluoro.

In some embodiments, the present invention provides a compound having the structure of Formula II:

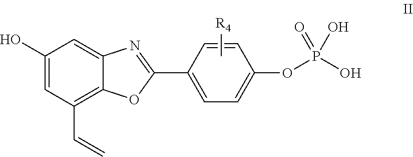

or a pharmaceutically acceptable salt thereof, wherein R₄ is as set forth above for Formula I.

In some embodiments, the present invention provides methods for treating or inhibiting a disease, disorder, or condition in a mammal, comprising the steps of: a) identifying a mammal having said disease, disorder, or condition; and b) administering to said mammal a therapeutically effective amount of a compound of Formula I or Formula III; and wherein said disease, disorder, or condition is selected from: an inflammatory disease, disorder, or condition; cancer; a cardiovascular disease, disorder, or condition; a cognitive disease, disorder, or condition; a disease, disorder, or condition of the skin; a neurodegenerative disease, disorder, or condition; diabetes; a disease, disorder, or condition associated with peri-menopause, menopause, or post-menopause; benign or malignant abnormal tissue growth including; and a disease, disorder or condition associated with a dysregulated systemic inflammatory response.

In some embodiments, a therapeutically effective amount of a compound of the present invention is administered parenterally. In some embodiments, the parenteral administration is subcutaneous, intravenous, or intramuscular.

In some embodiments, the inflammatory disease, disorder, or condition is selected from: prostatitis; interstitial cystitis; inflammatory bowel disease; Crohn's disease; ulcerative proctitis; colitis; arthritis; joint swelling or erosion; prostatic hypertrophy; asthma; pleurisy; and joint damage secondary to arthroscopic or surgical procedures. In some embodiments, the arthritis is rheumatoid arthritis or osteoarthritis. In some embodiments, the colitis is ulcerative colitis, indeterminate colitis, or infectious colitis.

In some embodiments, the cancer is selected from: uterine leiomyomas, breast cancer; endometrial cancer; endometrial cancer; benign breast disease; ovarian cancer; melanoma; prostrate cancer; colon cancer; and CNS cancers. In some embodiments the CNS cancer is glioma.

In some embodiments, the benign or malignant abnormal tissue growth is, glomerulosclerosis, uterine leiomyomas, scleroderma, fibromatosis, polycystic ovary syndrome, endometrial polyps, benign breast disease, or adenomyosis.

In some embodiments, the cardiovascular disease, disorder, or condition is selected from: aberrant cholesterol, triglyceride, Lp(a), or LDL levels; hypercholesteremia; hyperlipidemia; atherosclerosis; hypertension; peripheral vascular disease; restenosis; vasospasm; and vascular wall damage from cellular events leading toward immune mediated vascular damage.

In some embodiments, the cognitive disease, disorder, or condition is selected from: senile dementia; Alzheimer's disease; cognitive decline; stroke; anxiety; and free radical induced disease states; decreased libido; depression; insomnia; and schizophrenia.

In some embodiments, the disease, disorder, or condition of the skin is selected from psoriasis and dermatitis.

In some embodiments, the neurodegenerative disease, disorder, or condition is selected from: ischemia; reperfusion injury; multiple sclerosis; systemic lupus erythematosis; uveitis; and hemmorhagic shock.

Based on the results obtained in the standard pharmacological test procedures, compounds of this invention are expected to yield compounds that are estrogen receptor modulators useful in the treatment or inhibition of conditions, disorders, or disease states that are at least partially mediated by an estrogen deficiency or excess, or which may be treated or inhibited through the use of an estrogenic agent. Such compounds are particularly useful in treating a peri-menopausal, menopausal, or postmenopausal patient in which the levels of endogenous estrogens produced are greatly diminished. Menopause is generally defined as the last natural menstrual period and is characterized by the cessation of ovarian function, leading to the substantial diminution of circulating estrogen in the bloodstream. As used herein, menopause also includes conditions of decreased estrogen production that may be caused surgically, chemically, or by a disease state that leads to premature diminution or cessation of ovarian function.

In some embodiments, the disease, disorder, or condition associated with peri-menopause, menopause, or post-menopause is selected from: vaginal or vulvar atrophy; atrophic vaginitis; vaginal dryness; pruritus; dyspareunia; dysuria; frequent urination; urinary incontinence; urinary tract infections; vasomotor symptoms; endometriosis; dysfunctional uterine bleeding; and infertility; the disease, disorder or condition associated with a dysregulated systemic inflammatory response is selected from: sepsis; multiple organ failure; and septic shock.

In some embodiments, compounds of the present invention are administered in a therapeutically effective amount to treat or inhibit one or more symptoms associated with sepsis, multiple organ failure, or septic shock. In some embodiments, the symptoms associated with sepsis, multiple organ failure, or septic shock are selected from: hypothermia; hyperthermia; tachycardia; tachypnea or hyperventilation; and abnormal leukogram.

In some embodiments, compounds of the present invention may be used to prevent conception.

In some embodiments, compounds of the present invention are also useful in treating or inhibiting ocular disorders including cataracts, uveitis, and macular degeneration.

In some embodiments, compounds of the present invention are also useful in treating or inhibiting metabolic disorders; and bleeding disorders such as hereditary hemorrhagic telangiectasia, dysfunctional uterine bleeding, and combating hemorrhagic shock.

In some embodiments, compounds of the present invention are useful in disease states where amenorrhea is advantageous, such as leukemia, endometrial ablations, chronic renal or hepatic disease or coagulation diseases or disorders.

In some embodiments, a compound of the present invention is used in the preparation of a medicament for treating or inhibiting any of the diseases, disorders, or conditions described herein.

In some embodiments, the present invention provides processes for preparing compounds of Formula I as described above, comprising the step of: phosphorylating a compound of Formula IV:

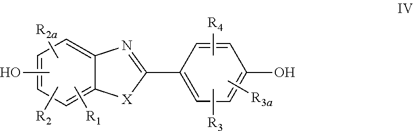

or a salt thereof with a phosphorylating reagent; wherein R₁, R₂, R₂ₐ, R₃, R₃ₐ, R₄, and X are as set forth above for Formula I.

In some embodiments, a compound of Formula I has the structure of Formula III:

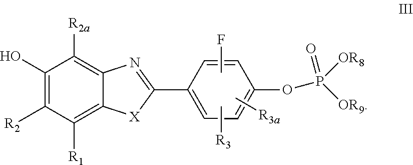

In some embodiments, a compound of Formula III has a structure of Formula IIIa:

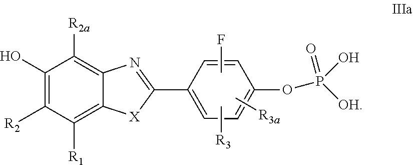

In some embodiments, a compound of Formula I has the structure of Formula IIIb:

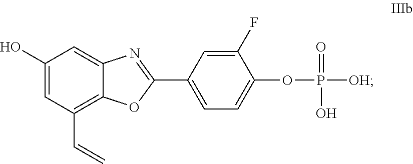

and a compound of Formula IV, has the structure of Formula IVa:

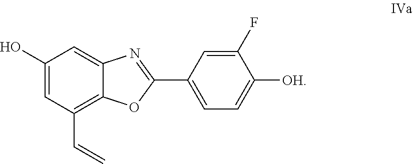

In some embodiments, the phosphorylating reagent comprises diethyl phosphate. In some embodiments, the diethyl phosphate is added to the reaction mixture over about 120 minutes. In some embodiments, the phosphoylrating reagent comprises diethyl chlorophosphate. In some embodiments, the phosphorylating agent is present at a value of about 1 molar equivalent to the compound of Formula IV. In some embodiments, the phosphorylating agent is present in molar excess to the compound of Formula IV.

In some embodiments, the phosphorylating reaction is performed in a solvent system. In some embodiments, the phosphorylating reaction is performed in a solvent system comprising a polar, aprotic organic solvent. In some embodiments, the solvent system comprises acetonitrile. In some embodiments, the phosphorylating reaction is carried out in the solvent system in the presence of a base. In some embodiments, the phosphorylating reaction is performed at a temperature of from about 15° C. to about 60° C.

In some embodiments, the present invention provides processes which further comprise isolating a compound of Formula I in its free acid form, or a salt thereof. Some embodiments further comprise the step of isolating a salt of the compound of Formula I, wherein the salt has the Formula Ib:

[R₁₁—O—PO₃⁻²]M

### Ib

wherein:

### R₁₁ is

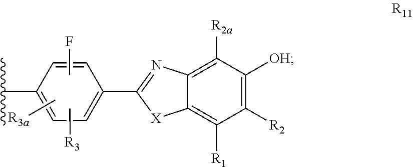

M is a Group I or II metal ion.

In some embodiments, R₁₁ has the Formula R₁₁ₐ:

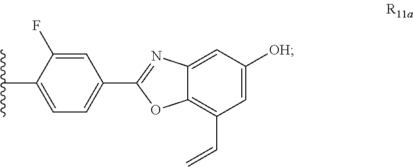

In some embodiments, the present invention provides processes which further comprise isolating a compound of Formula III in its free acid form, or a salt thereof. Some embodiments further comprise the step of isolating a salt of the compound of Formula III, wherein the salt has the Formula IIIc:

[R₁₁—O—PO₃⁻²]M

### IIIc

wherein:

### R₁₁ is

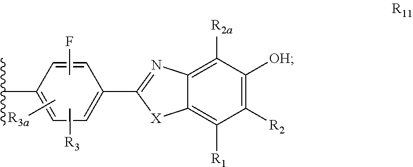

M is a Group I or II metal ion.

In some embodiments, R₁₁ has the Formula R₁₁ₐ:

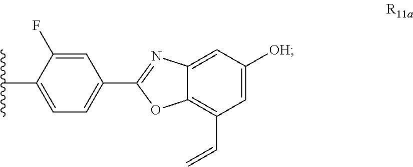

In some embodiments, the isolating of the compound in a free acid or salt form comprises the step of sufficiently removing hydrogen bromide from the reaction mixture. As used herein, “sufficiently removing” means removing hydrogen bromide to such an extent that when the residue obtained is contacted with an aqueous medium, it remains substantially free of a brominated by-product. As used herein, “substantially free of a brominated by-product” means no more than about 5% by weight, preferably no more than about 2% by weight, more preferably no more than about 1% by weight, more preferably no more than about 0.5% by weight, more preferably no more than about 0.1% by weight, more preferably no more than about 0.05% by weight, and more preferably no more than about 0.01% by weight of a given sample of compound contains a brominated by-product or a salt thereof.

In some embodiments, the reaction mixture is evaporated to sufficiently remove hydrogen bromide. In some embodiments, the reaction mixture is kept in vacuo to sufficiently remove hydrogen bromide. In some embodiments, the reaction mixture is evaporated in vacuo to sufficiently remove hydrogen bromide. In some embodiments, the reaction mixture is evaporated and kept in vacuo to sufficiently remove hydrogen bromide. In some embodiments, the reaction mixture is evaporated and then kept in vacuo to sufficiently remove hydrogen bromide. In some embodiments, the reaction mixture is kept in vacuo at from about 1 mm Hg to about 2 mm Hg to sufficiently remove hydrogen bromide. In some embodiments, the reaction mixture is kept in vacuo overnight to sufficiently remove hydrogen bromide. In some embodiments, overnight is from about 4 hours to about 16 hours. In some embodiments, overnight is from about 8 hours to about 14 hours. In some embodiments, overnight is about 12 hours.

In some embodiments, M is selected from Na⁺ ion, K⁺ ion, Li⁺ ion, Ca²⁺ ion, and Mg²⁺ ion. In some embodiments, M is Na⁺ ion. In some embodiments, M is K⁺ ion. In some embodiments, M is an NH₄⁺ ion rather than a Group I or Group II metal.

In some embodiments, the reaction mixture is treated with an alcohol to sufficiently remove hydrogen bromide. In some embodiments, the alcohol is methanol.

In some embodiments, the isolating of the compound in a free acid or salt form further comprises the step of treating the reaction mixture with a base. In some embodiments, the base is sodium hydroxide. In some embodiments, the base is aqueous sodium hydroxide. In some embodiments, the base is potassium hydroxide.

In some embodiments, the isolating of the compound of Formula I in a free acid or salt form optionally comprises one or more of distillation, distillation under reduced pressure, distillation further facilitated by adding a co-solvent, distillation under reduced pressure further facilitated by adding a co-solvent, evaporation of solvent followed by chromatography, triturating the salt with an organic solvent system comprising one or more polar organic solvents, high performance liquid chromatography (HPLC) and freeze drying.

In some embodiments, the isolating of the compound of Formula III in a free acid or salt form optionally comprises one or more of distillation, distillation under reduced pressure, distillation further facilitated by adding a co-solvent, distillation under reduced pressure further facilitated by adding a co-solvent, evaporation of solvent followed by chromatography, triturating the salt with an organic solvent system comprising one or more polar organic solvents, high performance liquid chromatography (HPLC) and freeze drying.

In some embodiments the present invention provides a compound that is 2-fluoro-4-(5-hydroxy-7-vinylbenzo[d]oxazol-2-yl)phenyl dihydrogen phosphate, or a pharmaceutically acceptable salt thereof. Some embodiments of the present invention provide a compound that is 2-(3-fluoro-4-hydroxyphenyl)-7-vinylbenzo[d]oxazol-5-yl dihydrogen phosphate, or a pharmaceutically acceptable salt thereof. Some embodiments of the present invention provide a compound that is:

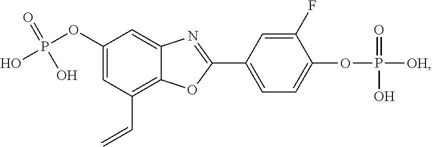

or a pharmaceutically acceptable salt thereof.

Some embodiments provide pharmaceutical compositions comprising 2-fluoro-4-(5-hydroxy-7-vinylbenzo[d]oxazol-2-yl)phenyl dihydrogen phosphate and optionally a pharmaceutically acceptable carrier. Some embodiments provide pharmaceutical compositions comprising 2-(3-fluoro-4-hydroxyphenyl)-7-vinylbenzo[d]oxazol-5-yl dihydrogen phosphate and optionally a pharmaceutically acceptable carrier. Some embodiments provide pharmaceutical compositions comprising:

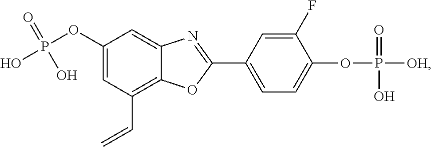

and optionally a pharmaceutically acceptable carrier. Some embodiments of the present invention provide a compound that is 2-fluoro-4-(5-(di-hydroxy)phosphoryloxy)-7-vinylbenzo[d]oxazol-2-ylphenyl dihydrogen phosphate, or a pharmaceutically acceptable salt thereof. Some embodiments provide a composition comprising 2-fluoro-4-(5-(di-hydroxy)phosphoryloxy)-7-vinylbenzo[dioxazol-2-ylphenyl dihydrogen phosphate, and optionally a pharmaceutically acceptable carrier. In some embodiments, the pharmaceutically acceptable carrier is an aqueous solvent.

In some embodiments, the present invention provides compositions comprising a compound of Formula II:

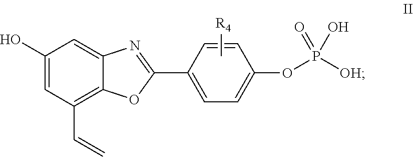

and optionally a pharmaceutically acceptable carrier.

In some embodiments, the present invention provides compositions comprising a compound of Formula V:

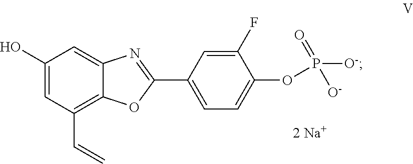

and optionally a pharmaceutically acceptable carrier. In some embodiments, the pharmaceutically acceptable carrier is an aqueous solvent.

In some embodiments, the present invention provides compositions comprising a compound of Formula VI:

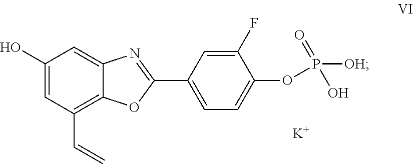

and optionally a pharmaceutically acceptable carrier.

In some embodiments, the present invention provides compositions comprising a compound of Formula VII:

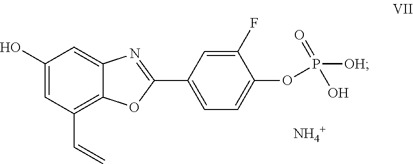

and optionally a pharmaceutically acceptable carrier.

In some embodiments, the present invention provides processes that are used to prepare compounds that are substantially free of compounds of Formula VIII or VIIIa:

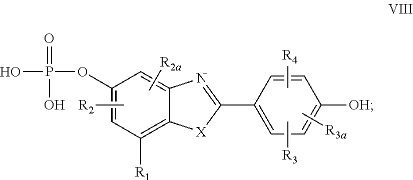

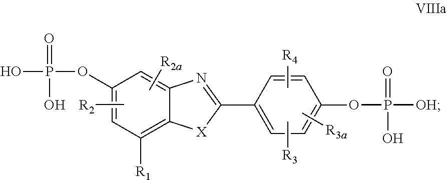

or salts thereof, wherein R₁, R₂, R₂ₐ, R₃, R₃ₐ, R₄, and X are as set forth above for Formula I. As used herein, the term “substantially free of compounds of Formula VIII or VIIIa” means that no more than about 5% by weight, preferably no more than about 2% by weight, more preferably no more that about 1% by weight, and more preferably no more than about 0.5% by weight of a given sample of compound has the Formula VIII or VIIIa or a salt thereof.

Generally, the phosphorylating reagent is employed in an amount of about 0.7 equivalent or more relative to the amount of compound of Formula IV or salt thereof, preferably about 1 molar equivalent relative to the amount of compound of Formula IV or salt thereof. In some embodiments the phosphorylating reagent is employed in an amount of about 2 molar equivalents or more relative to the amount of compound of Formula IV or salt thereof, or about 3 or more molar equivalents relative to the amount of compound of Formula IV or salt thereof.

Typically, the reaction of the compound of Formula IV and the phosphorylating reagent is performed in a solvent system, that can be a single solvent, or a mixture of solvents. In some embodiments, the phosphorylating reagent is a complex of diethyl phosphate and an amine. In some embodiments, the phosphorylating reagent is a complex of diethyl phosphate and acetonitrile. In some embodiments, phosphorylating reagent is a complex of diethyl chlorophosphate and an amine. In some embodiments, phosphorylating reagent is a complex of diethyl chlorophosphate and N,N-diisopropylethyl amine. A wide variety of suitable solvents can be employed, including polar organic solvents, preferably polar aprotic organic solvents, including those describe above. In some embodiments, the reaction is performed in a solvent system that includes or consists of acetonitrile. In some embodiments, the yield of a compound of Formula Ia or the salt thereof is greater than 50%, 55%, 60%, 65%, 75%, 80%, or 85%. In some embodiments, the yield of a compound of Formula lila or salt thereof is greater than 75%, 80%, 85%, 90%, or 95%.

The reaction of the compound of Formula IV and the phosphorylating reagent is performed at convenient temperature, for example from about 15° C. to about 60° C., preferably at from about 15° C. to about 27° C. Typically, the compound of Formula IV is dissolved in solvent, and the phosphorylating reagent is added slowly. The progress of the reaction can be monitored by a variety of techniques, for example by chromatographic techniques such as thin layer chromatography. The reaction between the compound of Formula IV and the phosphorylating reagent is typically complete after about 8 hours to about 2 days. In some embodiments, when the reaction between the compound of Formula IV and the phosphorylating reagent is complete, unreacted base is quenched, and the compound of Formula III or IIIa is isolated and obtained as the phosphate salt.

The salt can be isolated in relatively crude or in more pure form, depending upon the extent of purification. For example, in embodiments, the salt can be isolated by treating the reaction mixture with water to quench the base, filtering and evaporating the solvent to give a crude product, which can then be used as is in the optional deprotection step, or further purified by, for example, one or more of the foregoing techniques, such as silica chromatography.

It is appreciated that certain features of the invention, which are, for clarity, described in the context of separate embodiments, can also be provided in combination in a single embodiment. Conversely, various features of the invention which are, for brevity, described in the context of a single embodiment, can also be provided separately or in any suitable sub-combination.

The definitions set forth in this application are intended to clarify terms used throughout this application. The term “herein” means the entire application.

As used herein, the term “optionally substituted,” means that substitution is optional and therefore it is possible for the designated atom or moiety to be unsubstituted. In the event a substitution is desired then such substitution means that any number of hydrogens on the designated atom or moiety is replaced with a selection from the indicated group, provided that the normal valency of the designated atom or moiety is not exceeded, and that the substitution results in a stable compound. For example, if a methyl group (i.e., CH₃) is optionally substituted, then 3 hydrogens on the carbon atom can be replaced. Examples of suitable substituents include, but are not limited to: halogen, CN, NH₂, OH, SO, SO₂, COOH, OC₁₋₆ alkyl, CH₂OH, SO₂H, C₁₋₆ alkyl, OC₁₋₆ alkyl, C(═O)C₁₋₆ alkyl, C(═O)O—C₁₋₆ alkyl, C(═O)NH₂, C(═O)NHC₁₋₆ alkyl, C(═O)N(C₁₋₆ alkyl)2, SO₂C₁₋₆ alkyl, SO₂NH—C₁₋₆ alkyl, SO₂N(C₁₋₆ alkyl)₂, NH(C₁₋₆alkyl), N(C₁₋₆ alkyl)₂, NHC(═O)C₁₋₆ alkyl, NC(═O)(C₁₋₆ alkyl)₂, aryl, O-aryl, C(═O)-aryl, C(═O)O-aryl, C(═O)NH-aryl, C(═O)N(aryl)₂, SO₂-aryl, SO₂NH-aryl, SO₂N(aryl)₂, NH(aryl), N(aryl)₂, NC(═O)aryl, NC(═O)(aryl)₂, heterocyclyl, O-heterocyclyl, C(═O)-heterocyclyl, C(═O)O-heterocyclyl, C(═O)NH-heterocyclyl, C(═O)N(heterocyclyl)₂, SO₂-heterocyclyl, SO₂NH-heterocyclyl, SO₂N(heterocyclyl)₂, NH(heterocyclyl), N(heterocyclyl)₂, NC(═O)-heterocyclyl, and NC(═O)(heterocyclyl)₂, or any subset thereof.

As used herein, “floating substituent” is a substituent whose position on an aryl, cycloalkyl, or heterocyclyl ring is not fixed. In the below compound, for example, the R₃, R₃ₐ, and R₄ substituents are floating substituents, while the OA, OA′, R₁, R₂, and R₂ₐ substituents are fixed. As used herein, a floating substituent can only append from the particular ring with which it is shown to intersect. In compound Ia below, for example, R₄ can only append from the phenyl ring with which it is shown to intersect.

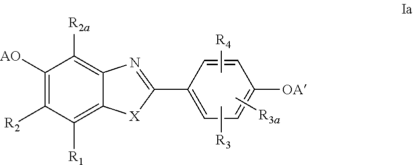

As used herein, “alkyl”, “alkylenyl” or “alkylene” used alone or as a suffix or prefix, is intended to include both branched and straight-chain saturated aliphatic hydrocarbon groups having from 1 to 12 carbon atoms or if a specified number of carbon atoms is provided then that specific number would be intended. For example “C₁₋₆ alkyl” denotes alkyl having 1, 2, 3, 4, 5 or 6 carbon atoms. Examples of alkyl include, but are not limited to, methyl, ethyl, n-propyl, i-propyl, n-butyl, i-butyl, sec-butyl, t-butyl, pentyl, and hexyl, or any subset thereof. As used herein, “C₁₋₃ alkyl”, whether a terminal substituent or an alkylene (or alkylenyl) group linking two substituents, is understood to specifically include both branched and straight-chain methyl, ethyl, and propyl.

As used herein, “alkenyl” refers to an alkyl group having one or more double carbon-carbon bonds. Example alkenyl groups include ethenyl, propenyl, cyclohexenyl, and the like. The term “alkenylenyl” refers to a divalent linking alkenyl group.

As used herein, “alkynyl” refers to an alkyl group having one or more triple carbon-carbon bonds. Example alkynyl groups include ethynyl, propynyl, and the like. The term “alkynylenyl” refers to a divalent linking alkynyl group.

As used herein, “aromatic” refers to hydrocarbyl groups having one or more polyunsaturated carbon rings having aromatic characters, (e.g., 4n+2 delocalized electrons) and comprising up to about 14 carbon atoms.

As used herein, “aryl” refers to monocyclic or polycyclic (e.g., having 2, 3 or 4 fused rings) aromatic hydrocarbons such as, for example, phenyl, naphthyl, anthracenyl, phenanthrenyl, indanyl, indenyl, and the like. In some embodiments, aryl groups have from 6 to about 20 carbon atoms. In some embodiments, aryl groups have from 6 to about 10 carbon atoms.

As used herein, “cycloalkyl” refers to non-aromatic cyclic hydrocarbons including cyclized alkyl, alkenyl, and alkynyl groups, having the specified number of carbon atoms (wherein the ring comprises 3 to 20 ring-forming carbon atoms). Cycloalkyl groups can include mono- or polycyclic (e.g., having 2, 3 or 4 fused or bridged rings) groups. Example cycloalkyl groups include cyclopropyl, cyclobutyl, cyclopentyl, cyclohexyl, cycloheptyl, cyclopentenyl, cyclohexenyl, cyclohexadienyl, cycioheptatrienyl, norbornyl, norpinyl, norcarnyl, adamantyl, and the like, or any subset thereof. The term “cycloalkyl” further includes fused or bridged polycyclic systems. Suitable cycloalkyls have from 3 to 10 carbon atoms in their ring structure, and more preferably have 3, 4, 5, and 6 carbons in the ring structure. For example, “C₃₋₆ cycloalkyl” denotes such groups as cyclopropyl, cyclobutyl, cyclopentyl, or cyclohexyl.

As used herein, the term “heterocyclyl” or “heterocyclic” or “heterocycle” refers to ring-containing monovalent and divalent structures having one or more heteroatoms, independently selected from N, O and S, as part of the ring structure and comprising from 3 to 20 atoms in the rings, or 3- to 7-membered rings. Heterocyclic groups may be saturated or partially saturated or unsaturated, containing one or more double bonds, and heterocyclic groups may contain more than one ring as in the case of polycyclic systems. The heterocyclic rings described herein may be substituted on carbon or on a heteroatom atom if the resulting compound is stable. If specifically noted, nitrogen in the heterocyclyl may optionally be quaternized. It is understood that when the total number of S and O atoms in the heterocyclyl exceeds 1, then these heteroatoms are not adjacent to one another.

Examples of heterocyclyls include, but are not limited to, 1H-indazole, 2-pyrrolidonyl, 2H, 6H-1, 5,2-dithiazinyl, 2H-pyrrolyl, 3H-indolyl, 4-piperidonyl, 4aH-carbazole, 4H-quinolizinyl, 6H-1, 2,5-thiadiazinyl, acridinyl, azabicyclo, azetidine, azepane, aziridine, azocinyl, benzimidazolyl, benzodioxol, benzofuranyl, benzothiofuranyl, benzothiophenyl, benzoxazolyl, benzthiazolyl, benzotriazolyl, benzotetrazolyl, benzisoxazolyl, benzisothiazolyl, benzimidazalonyl, carbazolyl, 4aH-carbazolyl, b-carbolinyl, chromanyl, chromenyl, cinnolinyl, diazepane, decahydroquinolinyl, 2H,6H-1,5,2-dithiazinyl, dioxolane, furyl, 2,3-dihydrofuran, 2,5-dihydrofuran, dihydrofuro[2,3-b]tetrahydrofuran, furanyl, furazanyl, homopiperidinyl, imidazolidine, imidazolidinyl, imidazolinyl, imidazolyl, 1H-indazolyl, indolenyl, indolinyl, indolizinyl, indolyl, isobenzofuranyl, isochromanyl, isoindazolyl, isoindolinyl, isoindolyl, isoquinolinyl, isothiazolyl, isoxazolyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxirane, oxazolidinylperimidinyl, phenanthridinyl, phenanthrolinyl, phenarsazinyl, phenazinyl, phenothiazinyl, phenoxathiinyl, phenoxazinyl, phthalazinyl, piperazinyl, piperidinyl, pteridinyl, piperidonyl, 4-piperidonyl, purinyl, pyranyl, pyrrolidinyl, pyrroline, pyrrolidine, pyrazinyl, pyrazolidinyl, pyrazolinyl, pyrazolyl, pyridazinyl, pyridooxazole, pyridoimidazole, pyridothiazole, pyridinyl, N-oxide-pyridinyl, pyridyl, pyrimidinyl, pyrrolidinyl, pyrrolidinyl dione, pyrrolinyl, pyrrolyl, pyridine, quinazolinyl, quinolinyl, 4H-quinolizinyl, quinoxalinyl, quinuclidinyl, carbolinyl, tetrahydrofuranyl, tetramethylpiperidinyl, tetrahydroquinoline, tetrahydroisoquinolinyl, thiophane, thiotetrahydroquinolinyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiopheneyl, thiirane, triazinyl, 1,2,3-triazolyl, 1,2,4-triazolyl, 1,2,5-triazolyl, 1,3,4-triazolyl, and xanthenyl, or any subset thereof.

As used herein, “heteroaryl” refers to an aromatic heterocycle (wherein the ring comprises up to about 20 ring-forming atoms) having at least one heteroatom ring member such as sulfur, oxygen, or nitrogen. Heteroaryl groups include monocyclic and polycyclic (e.g., having 2, 3 or 4 fused rings) systems. Examples of heteroaryl groups include without limitation, pyridyl (i.e., pyridinyl), pyrimidinyl, pyrazinyl, pyridazinyl, triazinyl, furyl (i.e. furanyl), quinolyl, isoquinolyl, thienyl, imidazolyl, thiazolyl, indolyl, pyrryl, oxazolyl, benzofuryl, benzothienyl, benzthiazolyl, isoxazolyl, pyrazolyl, triazolyl, tetrazolyl, indazolyl, 1,2,4-thiadiazolyl, isothiazolyl, benzothienyl, purinyl, carbazolyl, benzimidazolyl, indolinyl, and the like, or any subset thereof. In some embodiments, the heteroaryl group has from 1 to about 20 carbon atoms, and in further embodiments from about 1 to about 5, from about 1 to about 4, from about 1 to about 3, from about 1 to about 2, carbon atoms as ring-forming atoms. In some embodiments, the heteroaryl group has 1 to about 4, 1 to about 3, or 1 to 2 heteroatoms. In some embodiments, the heteroaryl group has 1 heteroatom.

As used herein, “heterocycloalkyl” refers to non-aromatic heterocycles (wherein the ring comprises about 3 to about 20 ring-forming atoms) including cyclized alkyl, alkenyl, and alkynyl groups where one or more of the ring-forming carbon atoms is replaced by a heteroatom such as an O, N, or S atom. Hetercycloalkyl groups can be mono or polycyclic (e.g., fused-, bridged- and spiro-systems). Suitable “heterocycloalkyl” groups include morpholino, thiomorpholino, piperazinyl, tetrahydrofuranyl, tetrahydrothienyl, 2,3-dihydrobenzofuryl, 1,3-benzodioxole, benzo-1,4-dioxane, piperidinyl, pyrrolidinyl, isoxazolidinyl, isothiazolidinyl, pyrazolidinyl, oxazolidinyl, thiazolidinyl, imidazolidinyl, and the like. Ring-forming carbon atoms and heteroatoms of a heterocycloalkyl group can be optionally substituted by oxo or sulfido. Also included in the definition of heterocycloalkyl are moieties that have one or more aromatic rings fused (i.e., having a bond in common with) to the nonaromatic heterocyclic ring, for example phthalimidyl, naphthalimidyl, and benzo derivatives of heterocycles such as indolene and isoindolene groups. In some embodiments, the heterocycloalkyl group has from 1 to about 20 carbon atoms, and in further embodiments from about 3 to about 20 carbon atoms. In some embodiments, the heterocycloalkyl group contains 3 to about 14, 3 to about 7, or 5 to 6 ring-forming atoms. In some embodiments, the heterocycloalkyl group has 1 to about 4, 1 to about 3, or 1 to 2 heteroatoms. In some embodiments, the heterocycloalkyl group contains 0 to 3 double bonds. In some embodiments, the heterocycloalkyl group contains 0 to 2 triple bonds.

As used herein, “alkoxy” or “alkyloxy” represents an alkyl group as defined above with the indicated number of carbon atoms attached through an oxygen bridge. Examples of alkoxy include, but are not limited to, methoxy, ethoxy, n-propoxy, isopropoxy, n-butoxy, isobutoxy, t-butoxy, n-pentoxy, isopentoxy, cyclopropylmethoxy, allyloxy and propargyloxy, or any subset thereof. Similarly, “alkylthio” or “thioalkoxy” represent an alkyl group as defined above with the indicated number of carbon atoms attached through a sulphur bridge.

As used herein, “halo” or “halogen” includes fluoro, chloro, bromo, and iodo, or any subset thereof.

As used herein, “haloalkyl” refers to an alkyl group having one or more halogen substituents. Example haloalkyl groups include CF₃, C₂F₅, CH₂CF₃, CHF₂, CCl₃, CHCl₂, C₂Cl₅, and the like, or any subset thereof. The term “perhaloalkyl” is intended to denote an alkyl group in which all of the hydrogen atoms are replaced with halogen atoms. One example of perhaloalkyl is CH₃ or CF₃. The term “perfluoroalkyl” is intended to denote an alkyl group in which all of the hydrogen atoms are replaced with fluorine atoms. One example of perhaloalkyl is CF₃ (i.e., trifluoromethyl).

As used here, “haloalkoxy” refers to an —O-haloalkyl group. An example haloalkoxy group is OCF₃.

As used herein, “aryloxy” refers to O-aryl. An example heteroaryloxy is phenoxy. As used herein, the phrase “protecting group” means temporary substituents which protect a potentially reactive functional group from undesired chemical transformations. Examples of such protecting groups include esters of phosphoric acids, esters of carboxylic acids, silyl ethers of alcohols, and acetals and ketals of aldehydes and ketones respectively. The field of protecting group chemistry has been reviewed (Greene, T. W.; Wuts, P. G. M. Protective Groups in Organic Synthesis, 3^{rd} ed.; Wiley: New York, 1999).

As used herein, “pharmaceutically acceptable salts” refer to derivatives of the disclosed compounds wherein the parent compound is modified by making acid or base salts thereof (i.e., also include counterions). Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, phosphoric, and the like; and the salts prepared from organic acids such as lactic, maleic, citric, benzoic, methanesulfonic, and the like.

The pharmaceutically acceptable salts of the present invention can be synthesized from the parent compound that contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; nonaqueous media like ether, ethyl acetate, ethanol, isopropanol, or acetonitrile can be used.

A variety of compounds in the present invention may exist in particular stereoisomeric forms. The present invention takes into account all such compounds, including cis- and trans isomers, R- and S-enantiomers, diastereomers, (D)-isomers, (L)-isomers, the racemic mixtures thereof, and other mixtures thereof, as being covered within the scope of this invention. Additional asymmetric carbon atoms may be present in a substituent such as an alkyl group. All such isomers, as well as mixtures thereof, are intended to be included in this invention. The compounds herein described may have asymmetric centers. Compounds of the present invention containing an asymmetrically substituted atom may be isolated in optically active or racemic forms. It is well known in the art how to prepare optically active forms, such as by resolution of racemic forms or by synthesis from optically active starting materials. When required, separation of the racemic material can be achieved by methods known in the art.

Many stereoisomers of olefins, C═N double bonds, and the like can also be present in the compounds described herein, and all such stable isomers are contemplated in the present invention. Cis and trans isomers of the compounds of the present invention are described and may be isolated as a mixture of isomers or as separated isomeric forms. All chiral, diastereomeric, racemic forms and all stereoisomeric forms of a structure are intended, unless the specific stereochemistry or isomeric form is specifically indicated.

The phosphate derivatives described herein are prodrugs that are convertible in vivo or in vitro into one of the parent compounds. Typically, at least one of the biological activities of compound will be reduced in the prodrug form of the compound, and can be activated by conversion of the prodrug to release the compound or a metabolite of it. Some prodrugs are esters of the active compound (e.g., a physiologically acceptable metabolically labile ester). During metabolism, the ester group (—C(═O)OR) is cleaved to yield the active drug. Such esters may be formed by esterification, for example, of any of the carboxylic acid groups (—C(═O)OH) in the parent compound, with, where appropriate, prior protection of any other reactive groups present in the parent compound, followed by deprotection if required.

Examples of such metabolically labile esters include those of the formula —C(═O)OR wherein R is: C₁₋₇alkyl (e.g., Me, Et, -nPr, -iPr, -nBu, -sBu, -iBu, tBu); C₁₋₇aminoalkyl (e.g., aminoethyl; 2-(N,N-diethylamino)ethyl; 2(4morpholino)ethyl); and acyloxy-C₁₋₇alkyl (e.g., acyloxymethyl; acyloxyethyl; pivaloyloxymethyl; acetoxymethyl; 1acetoxyethyl; 1-(1-methoxy-1-methyl)ethyl-carbonyloxyethyl; 1-(benzoyloxy)ethyl; isopropoxy-carbonyloxymethyl; 1-isopropoxy-carbonyloxyethyl; cyclohexyl-carbonyloxymethyl; 1-cyclohexyl-carbonyloxyethyl; cyclohexyloxy-carbonyloxymethyl; 1-cyclohexyloxy-carbonyloxyethyl; (4-tetrahydropyranyloxy)carbonyloxymethyl; 1-(4-tetrahydropyranyloxy)carbonyloxyethyl; (4-tetrahydropyranyl)carbonyloxymethyl; and 1-(4-tetrahydropyranyl)carbonyloxyethyl), or any subset thereof.

Phosphorates (phosphoric acid esters) can be formed between a hydroxyl group present in the compound and an apporopriate phosphoric acid reaction partner, using techniques known in the art. Some prodrugs are phosphorates of the active compound having a hydroxyl group (HOR), for example, a physiologically acceptable metabolically labile phosphorite. During metabolism (in the presence of alkaline phosphatase), the P—OR bond of a compound having the formula of P(═O)(OH)₂OR is cleaved to yield the active drug (HOR).

Also, some prodrugs are activated enzymatically to yield the active compound, or a compound which, upon further chemical reaction, yields the active compound.

Where the compounds contain chiral centres, all individual optical forms such as enantiomers, epimers and diastereoisomers, as well as racemic mixtures of the compounds are within the scope of the invention.

Compounds may exist in a number of tautomeric forms and references to compounds include all such forms. For the avoidance of doubt, where a compound can exist in one of several tautomeric forms and only one is specifically described or shown, all others are nevertheless embraced by the scope of this invention. As used herein, “tautomer” means other structural isomers that exist in equilibrium resulting from the migration of a hydrogen atom. For example, keto-enol tautomerism where the resulting compound has the properties of both a ketone and an unsturated alcohol.

Compounds of the present invention also include pharmaceutically acceptable salts and tautomers of the compounds of any of the formulas described herein. Compounds of the present invention further include hydrates and solvates.

### Synthesis

In some embodiments, compounds of the present invention are derivatives that possess one or more appended phosphate (i.e., —P(O)(OH)₂) groups.

Compounds of the present invention can be prepared, for example, using the reaction pathways and techniques as described below in Scheme I.

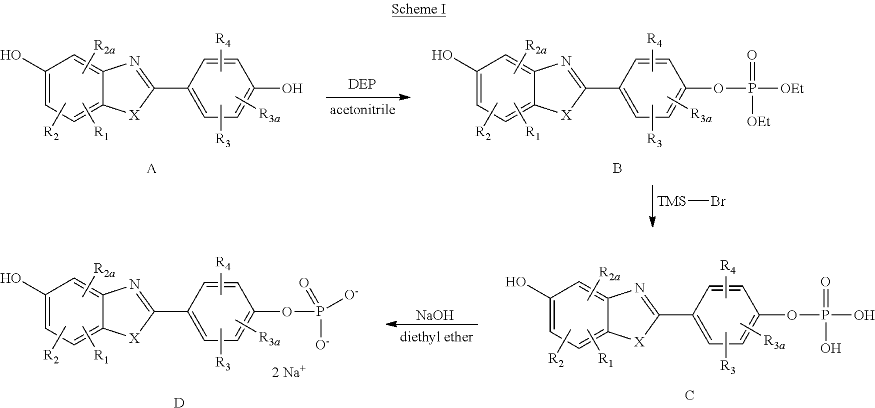

The preparation of compounds of Formula A are described in U.S. Pat. No. 6,794,403, hereby incorporated by reference in its entirety.

As can be seen in Scheme I, the starting material of Formula A has two reactive hydroxyl groups and the present invention surprisingly provides a convenient route for the preparation of the mono-phosphate product of Formula D which is substantially free of the di-phosphate byproduct or of the product of Formula VIII or VIIIa above (mono-phosphated at the fused ring system hydroxyl group) or their salts.

### Dosage and Formulation

When administered for the treatment or inhibition of a particular disease state or disorder, it is understood that the effective dosage may vary depending upon the particular compound utilized, the mode of administration, the condition, and severity thereof, of the condition being treated, as well as the various physical factors related to the individual being treated. Effective administration of the compounds of this invention may be given at an oral or intravenous dose of from about 0.1 mg/day to about 1,000 mg/day. Preferably, administration will be from about 10 mg/day to about 600 mg/day, more preferably from about 50 mg/day to about 600 mg/day, in a single dose or in two or more divided doses. The projected daily dosages are expected to vary with route of administration.

In some embodiments, compounds of the present invention are administered intravenously at a dose of 0.3 mg/kg. In some embodiments, compounds of the present invention are administered intravenously at a dose of 1 mg/kg. In some embodiments, compounds of the present invention are administered intravenously at a dose of 3 mg/kg. In some embodiments, compounds of the present invention are administered intravenously every 24 hours.

Such doses may be administered in any manner useful in directing the active compounds herein to the recipient's bloodstream, including orally, via implants, parentally (including intravenous, intraperitoneal, intraarticularly and subcutaneous injections), rectally, intranasally, topically, ocularly (via eye drops), vaginally, and transdermally.

Oral formulations containing the compounds of this invention may comprise any conventionally used oral forms, including tablets, capsules, buccal forms, troches, lozenges and oral liquids, suspensions or solutions. Capsules may contain mixtures of the active compound(s) with inert fillers and/or diluents such as the pharmaceutically acceptable starches (e.g., corn, potato or tapioca starch), sugars, artificial sweetening agents, powdered celluloses, such as crystalline and microcrystalline celluloses, flours, gelatins, gums, etc. Useful tablet formulations may be made by conventional compression and wet granulation or dry granulation methods, and utilize pharmaceutically acceptable diluents, binding agents, lubricants, disintegrants, surface modifying agents (including surfactants), suspending or stabilizing agents, including, but not limited to, magnesium stearate, stearic acid, talc, sodium lauryl sulfate, microcrystalline cellulose, carboxymethylcellulose calcium, polyvinylpyrrolidone, gelatin, alginic acid, acacia gum, xanthan gum, sodium citrate, complex silicates, calcium carbonate, glycine, dextrin, sucrose, sorbitol, dicalcium phosphate, calcium sulfate, lactose, kaolin, mannitol, sodium chloride, talc, dry starches and powdered sugar. Preferred surface modifying agents include nonionic and anionic surface modifying agents. Representative examples of surface modifying agents include, but are not limited to, poloxamer 188, benzalkonium chloride, calcium stearate, cetostearl alcohol, cetomacrogol emulsifying wax, sorbitan esters, colloidol silicon dioxide, phosphates, sodium dodecylsulfate, magnesium aluminum silicate, and triethanolamine. Oral formulations herein may utilize standard delay or time release formulations to alter the absorption of the active compound(s). In some embodiments, the oral formulation may also consist of administering the active ingredient in water or a fruit juice, containing appropriate solubilizers or emulsifiers as needed.

In some embodiments, it may be desirable to administer the compounds directly to the airways in the form of an aerosol.

In some embodiments, compounds of the present invention may also be administered parenterally or intraperitoneally. Solutions or suspensions of these active compounds as a free base or pharmacologically acceptable salt can be prepared in water suitably mixed with a surfactant such as hydroxy-propylcellulose. Dispersions can also be prepared in glycerol, liquid polyethylene glycols and mixtures thereof in oils. Under ordinary conditions of storage and use, these preparations contain a preservative to inhibit the growth of microorganisms.

The pharmaceutical forms suitable for injectable use include sterile aqueous solutions or dispersions and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), suitable mixtures thereof, and vegetable oils.

For the purposes of this disclosure, transdermal administrations are understood to include all administrations across the surface of the body and the inner linings of bodily passages including epithelial and mucosal tissues. Such administrations may be carried out using the present compounds, or pharmaceutically acceptable salts thereof, in lotions, creams, foams, patches, suspensions, solutions, and suppositories (rectal and vaginal).

Transdermal administration may be accomplished through the use of a transdermal patch containing the active compound and a carrier that is inert to the active compound, is non toxic to the skin, and allows delivery of the agent for systemic absorption into the blood stream via the skin. The carrier may take any number of forms such as creams and ointments, pastes, gels, and occlusive devices. The creams and ointments may be viscous liquid or semisolid emulsions of either the oil-in-water or water-in-oil type. Pastes comprised of absorptive powders dispersed in petroleum or hydrophilic petroleum containing the active ingredient may also be suitable. A variety of occlusive devices may be used to release the active ingredient into the blood stream such as a semi-permeable membrane covering a reservoir containing the active ingredient with or without a carrier, or a matrix containing the active ingredient. Other occlusive devices are known in the literature.

Suppository formulations may be made from traditional materials, including cocoa butter, with or without the addition of waxes to alter the suppository's melting point, and glycerin. Water soluble suppository bases, such as polyethylene glycols of various molecular weights, may also be used.

The compounds of the present invention are substituted benzoxazole estrogenic agents, which have been derivatized to possess one or more appended moieties. After administration of the derivatized compound, the appended moieties are removed by endogenous enzymes to provide the underivatized compound. Such compounds are referred to here as metabolites of the compounds of the invention.

As used in accordance with this invention, the term “providing,” with respect to providing a compound or substance covered by this invention, means either directly administering such a compound or substance, or administering a prodrug, derivative, or analog that will form the effective amount of the compound or substance within the body.

As used in accordance with this invention, the term “ERβ selective ligand” means that the binding affinity (as measured by IC₅₀, where the IC₅₀ of 17β-estradiol is not more than 3 fold different between ERα and ERβ) of the ligand to ERβ is at least about 10 times greater than its binding affinity to ERα in a standard pharmacological test procedure that measures the binding affinities to ERα and ERβ. It is preferred that the ERβ selective ligand will have a binding affinity to ERβ that is at least about 20 times greater than its binding affinity to ERα. It is more preferred that the ERβ selective ligand will have a binding affinity to ERα that is at least about 50 times greater than its binding affinity to ERα. It is further preferred that the ERβ selective ligand is non-uterotrophic and non-mammotrophic.

As used in accordance with this invention, the term “non-uterotrophic” means producing an increase in wet uterine weight in a standard pharmacological test procedure of less than about 50% of the uterine weight increase observed for a maximally efficacious dose of 17β-estradiol or 17α-ethinyl-17β-estradiol in the same procedure. It is preferred that the increase in wet uterine weight will be less than about 25% of that observed for estradiol, and more preferred that the increase in wet uterine weight will be less than about 10% of that observed for estradiol. It is most preferred that the non-uterotrophic ERβ selective ligand will not increase wet uterine weight significantly (p>0.05) compared with a control that is devoid of uterotrophic activity (e.g., vehicle).

As used in accordance with this invention, the term “non-mammotrophic” means having activity that is <10% as efficacious as 17beta-estradiol at facilitating the development of lobular-alveolar end buds as assessed by histological examination. Examples of such determination by histological examination are well known in the art. See, for example, Harris, H. A., et al., Endocrinology 144(10) 4241-4249 (2003); Mulac-Jericevic, B., et al., Proc. Natl. Acad. Sci, 100 (17) 9744-9749 (2003); Bocchinfuso, W. P., et al., Endocrinology 141(8) 2982-2994 (2002); and Lewis, B. C., et al., Toxicological Sciences 62, 46-53 (2001), each of which is incorporated by reference herein in its entirety.

Compounds of the invention can also include all isotopes of atoms occurring in the intermediates or final compounds. Isotopes include those atoms having the same atomic number but different mass numbers. For example, isotopes of hydrogen include tritium and deuterium.

The invention will be described in greater detail by way of specific examples. The following example is offered for illustrative purposes, and are not intended to limit the invention in any manner. Those of skill in the art will readily recognize a variety of noncritical parameters which can be changed or modified to yield essentially the same results.

### EXAMPLES

### Example 1

### Sodium 2-fluoro-4-(5-hydroxy-7-vinylbenzo[d]oxazol-2-yl)phenyl phosphate

### Step 1: Diethyl 2-fluoro-4-(5-hydroxy-7-vinylbenzoldfoxazol-2-yl)phenyl phosphate

A solution of diethyl phosphate (10 mmole, 1381 mg) in acetonitrile (145 mL) was added dropwise over 120 minutes to a solution of 2-(3-fluoro-4-hydroxyphenyl)-7-vinyl-1,3-benzoxazol-5-ol (10 mmole, 2712 mg) in anhydrous tetrahydrofuran (145 mL) containing N,N-diisopropylethylamine (20 mmole, 2585 mg), 4-di(methylamino)pyridine (1 mmole, 122.1 mg) and carbon tetrachloride (50 mmole, 7691 mg) at ambient temperature. After stirring the reaction mixture for 22 hours at ambient temperature the mixture was concentrated in vacuo to ˜⅕ of its volume then water (200 mL) was added and the product extracted with diethyl ether (3×70 mL). The combined organic phase was washed with brine, separated, dried over magnesium sulfate, filtered and the filtrate evaporated in vacuo. The obtained solid residue was subjected to high vacuum (2 Torr) for 24 hours yielding 3800 mg (93%) of the title compound as an off-white solid; m.p. 95-7° C. MS [ES]⁺: m+H 408.1. MS [ES]⁻: m−H 406.1.

### Step 2: 2-Fluoro-4-(5-hydroxy-7-vinylbenzo[d]oxazol-2-yl)phenyl dihydrogen phosphate

Under dry nitrogen a solution of the starting ester (14.24 mmole, 5800 mg) in 1,2-dichloroethane (350 mL) was treated at once with bromotrimethylsilane (100 mmole, 15310 mg). The reaction mixture was refluxed for 62 minutes, cooled to ambient temperature and then evaporated in vacuo to dryness. The solid residue was subjected to sodium hydroxide (1N, 28.4 mmole, 28.4 mL) diluted with water (100 mL) and stirred at ambient temperature for 20 minutes. The aqueous solution was extracted with diethyl ether (150 mL). The layers were separated and the aqueous phase lyophilized to give 7000 mg of the desired title compound (contains >15% water) as a white powder; m.p. contracts >160° C. MS [ES]⁻: m−H 350.

### Example 2

### 2-fluoro-4-(5-hydroxy-7-vinylbenzo[d]oxazol-2-yl)phenyl phosphoric acid

### Step 1: Diethyl 2-fluoro-4-(5-hydroxy-7-vinylbenzo[d]oxazol-2-yl)phenyl phosphate

To the suspension of 2-(3-fluoro-4-hydroxyphenyl)-7-vinyl-1,3-benzoxazole-5-ol (99 g, 0.36 mol) in dry acetonitrile (1 L) was added N,N-diisopropylethyl amine(93 g, 0.72 mol, 2 eq.) followed by diethyl chlorophosphate (67.2 g, 0.39 mol, 1.1 eq.) by dropwise addition over 1 h at the room temperature. The reaction mixture was stirred for 16 h and concentrated to min. The oily residue was triturated with water (300 ml×3) and filtered. The wet cake was slurred in ether (300 ml), filtered, thoroughly was washed with ether (200 mL×3) and dried in vacuum oven at 63° C. for 14 h to give 96% pure white powder (90.7 g, 0.22 mol). Yield 62%. M.p.134-136° C. MS/ES [M+H] 408.1

### Step 2: 2-Fluoro-4-(5-hydroxy-7-vinylbenzo[d]oxazol-2-y!)phenyl dihydrogen phosphate

The clear solution of diethyl 2-fluoro-4-(5-hydroxy-7-vinylbenzo[d]oxazol-2-yl)phenyl phosphate (90.5 g, 0.22 mol) and bromo trimethylsilane (144 g, 0.94 mol, 4.27 eq.) in methylene chloride (1L) was heated at gentle reflux for 23 h. The reaction mixture was concentrated in vacua, the residue was stirred in methanol (300 mL) for 20 min. To the formed thick slurry was added t-butyl methyl ether (200 ml) and stirred for additional 20 min and filtered. The cake was washed with ether (200 mL×3) and air dried to give 98% pure yellow solids (73 g, 0.2 mol). Yield 94%.

MS/ES: [M−H] 350.1

### Example 3

### Potassium 2-fluoro-4-(5-hydroxy-7-vinylbenzo[d]oxazol-2-yl)phenyl phosphate

To the slurry of 2-fluoro-4-(5-hydroxy-7-vinylbenzo[d]oxazol-2-yl)phenyl dihydrogen phosphate (72.8 g, 0.2 mol) in dry ethanol (1.1 L) was added solution of potassium hydroxide in dry ethanol (11.7 g in 500 mL ethanol, 0.2 mol). To the reaction mixture stirred for 40 min., water (15 mL, 3 eq.) was added and stirred additionally for 15 min and filtered. The cake was washed with ethanol and air dried to give 99% pure crystalline white solids (67.8 g, 0.17 mop. Yield 85%. M.p.245° C. MS/ES: [M−H] 350.

### Example 4

### Ammonium 2-fluoro-4-(5-hydroxy-7-vinylbenzo[d]oxazol-2-yl)phenyl phosphate

To the slurry of 2-fluoro-4-(5-hydroxy-7-vinylbenzo[d]oxazol-2-yl)phenyl dihydrogen phosphate (1.5 g, 4.28 mmol) was added 2M methanolic ammonia (2.15 mL, 4.30 mmol). The mixture was stirred for 35 min and filtered, washed with ethanol and air dried to give 98% pure crystalline white solids (1 g, 2.68 mol). Yield 62%. MS/ES: [M−H] 350.

### Example 5

### 2[3-Fluoro-4-(phosphonooxy)phenyl]-7-vinyl-1,3-benzoxazol-5-yl dihydrogen phosphate

### Step 1: 2-{4-[(Diethoxyphosphoryl)oxy]-3-fluorophenyl}-7-vinyl-1,3-benzoxazol-5-yl diethyl phosphate

A solution of diethyl phosphate (10 mmole, 1381 mg) in acetonitrile (72 mL) was added dropwise over 120 minutes to a solution of ERB-041 (4 mmole, 1085 mg) in anhydrous tetrahydrofuran (72 mL) containing N,N-diisopropylethylamine (15 mmole, 1938 mg), 4-di(methylamino)pyridine (0.75 mmole, 92 mg) and carbon tetrachloride (38 mmole, 5844 mg) at ambient temperature. After stirring the reaction mixture for 6 hours at ambient temperature water (100 mL) was added, concentrated in vacuo and the product extracted with ethyl acetate (2×70 mL). The combined organic phase was washed with brine, separated, dried over magnesium sulfate, filtered and the filtrate evaporated in vacuo. The obtained oily residue was subjected to HPLC purification. Elution with 3% methanol in hexane containing 10% dichloromethane on a primesphere CN column (5×15 cm) furnished after evaporation of the solvent 1350 mg (62%) of the title compound as a dense colorless oil in 99.6% purity. MS [ES]⁺: m+H 544.1.

### Step 2: 2-[3-Fluoro-4-(phosphonooxy)phenyl]-7-vinyl-1,3-benzoxazol-5-yl dihydrogen phosphate

A mixture of the starting cheater (1.65 mmole, 900 mg) and bromo trimethylsilane (26.4 mmole, 4042 mg) was stirred at ambient temperature for 72 hours. The reaction mixture was evaporated in vacuo and the residue partitioned between water (80 mL) and ether (100 mL). The bi-layer system was stirred for 30 minutes and the aqueous phase separated. Tributylamine (16.5 mmole, 3058 mg) was added to the aqueous layer and stirred for 15 minutes. The organic phase was separated and the aqueous phase was concentrated in vacuo. The residue was lyophilized overnight yielding 1300 mg (98%) of the title compound as off-white microcrystals; m.p. >180° C. (Decomposition). MS [ES]⁺: m+H 802.1.

### Example 6

The cecal ligation puncture (CLP) procedure was derived from a modification of standard methods previously described. Female, non-pregnant, specific pathogen-free, Albino BALBc mice (20-25 gm) were used in this experiment. The test compound (n=8 in each group) or corn oil control (n=12) were randomized to a treatment protocol consisting of 50 mg/kg of either the test compound or similar volume of corn oil at the time of CLP (time 0) and 24 and 48 hours following surgery. Animals were allowed to eat and drink sterile water on an ad libitum basis until 12 h before surgery when food was withheld until after the surgical procedure.

Anesthetized animals (methoxyflurane, Mallinckodt Veterninary Inc., Mundeline, Ill.) had the abdominal skin shaved and a midline, abdominal incision was made. The cecum was exteriorized and distended using intraluminal contents from the colon. The cecum was then ligated below the ileocecal valve and punctured twice using a 23-gauge sterile needle. The cecum was returned to the peritoneal cavity and the transversalis fascia was closed along with the skin incision. The skin incision was covered with topical bacitracin ointment. The animals were given a single intravenous dose of trovafloxacin (20 mg/kg IM) immediately post-operatively and a 1-mL subcutaneous dose of PBS was given as fluid resuscitation. The animals were observed for 7 days on a daily basis and deaths were recorded. All animals underwent necropsy examination for histological evidence of organ injury, pathology scoring of intestinal mucosa and quantitative bacteriology.

Differences in survival time between groups were analyzed by Kaplan-Meier survival plots and log-rank testing. Other parameters were measured using a Mann-Whitney U-Test for 2 groups or the Kruskal-Wallis one way analysis of variance for 3 or more groups. A p value of <0.05 was considered significant. All data is presented as mean and standard deviation.

The root compound (ERB-041) was dosed IV in the mCLP model, at 1 and 3 mg/kg at 24, 48 and 72 hours after induction of peritonitis. This therapeutic IV administration in mCLP confirmed attenuation of lung injury and cytokine/chemokine responses and attenuation of the subsequent clinical course, respectively.⁵ achieving 7-day survival of 95 and 90% in the 3 and 1 mg/kg groups.

Intravenous treatment with the test compound, at 0.3, 1, and 3 mg/kg given at 24, 48 and 72 hours after surgery, demonstrated a clear dose response trend for increasing 7-day survival, 10% survival in the vehicle, 40% in the 0.3mg/kg group and 60% survival in both the 1 and 3 mg/kg groups. The 1 and 3 mg/kg doses were different from vehicle (0.05).

### Example 7

As exemplified by Table 1 below, the mono-phosphate pro-drug of the root compound exhibited increased aqueous solubility relative to the root compound. This improved solubility allowed administration of the mono-phosphate derivative in a phosphate buffered saline vehicle. Further, the mono-phosphate derivative demonstrated reasonable IV pharmacokinetics, and demonstrated efficacy in mCLP, with a minimally effective does of 1 mg/kg IV.

TABLE 1

| | | Mono-Phosphate |
|---|---|---|
| Properties (in vitro) | ERB 041 | derivative of ERB 041 |
| clogP | 3.9 | 2.44 |
| Solubility (pH 7.4) | 4 μg/mL | >100 μg/mL |

### Example 8

The mono-phosphate derivatives are converted in vivo to the parent compounds by the activity of phosphatases. A mono-phosphate derivative and its hydrolysis product, root compound, were observed in the plasma of early time points taken from pharmacokinetic studies of the mono-phosphate derivative conducted in mice, rats and monkeys.

In this study, 5, 15, 30 min, 1, 2, 4, 6, 8 and 24 hr plasma samples were collected following a single IV dose of the mono-phosphate derivative (1.3 and 3.9 mg/kg) to mice, rats and monkeys. Equal volume (100 μL) of samples from 5, 15, 30 min and 1, 2, 4 hr and 6, 8, 24 hr time points from each subject (n=3) were pooled from the two dosing groups. Aliquots (900 μL) of pooled plasma were mixed with 2700 μL of cold acetonitrile and centrifuged at 3200×g for 10 minutes. The resulting supernatants were transferred to clean tubes and evaporated to dryness in a TurboVap LV at ambient temperature under a slow stream of nitrogen. The dried extracts were reconstituted with 300 μL of 20:80 acetonitrile:water (v/v). The samples were then analyzed by LC/UV/MS for metabolite profiling and characterization. LC-ESI/MS in negative ionization mode was used to acquire mass spectral data. Conversion of the phosphate derivatives to the root compound in plasma and whole blood was species specific and was substantial in hepatocytes from all species tested (mice, rat, monkey, human). Proposed metabolites of the phosphate prodrug in ASD mouse, rat, and monkey plasma are shown in Scheme 2 below and further characterized in Table 2, also below.

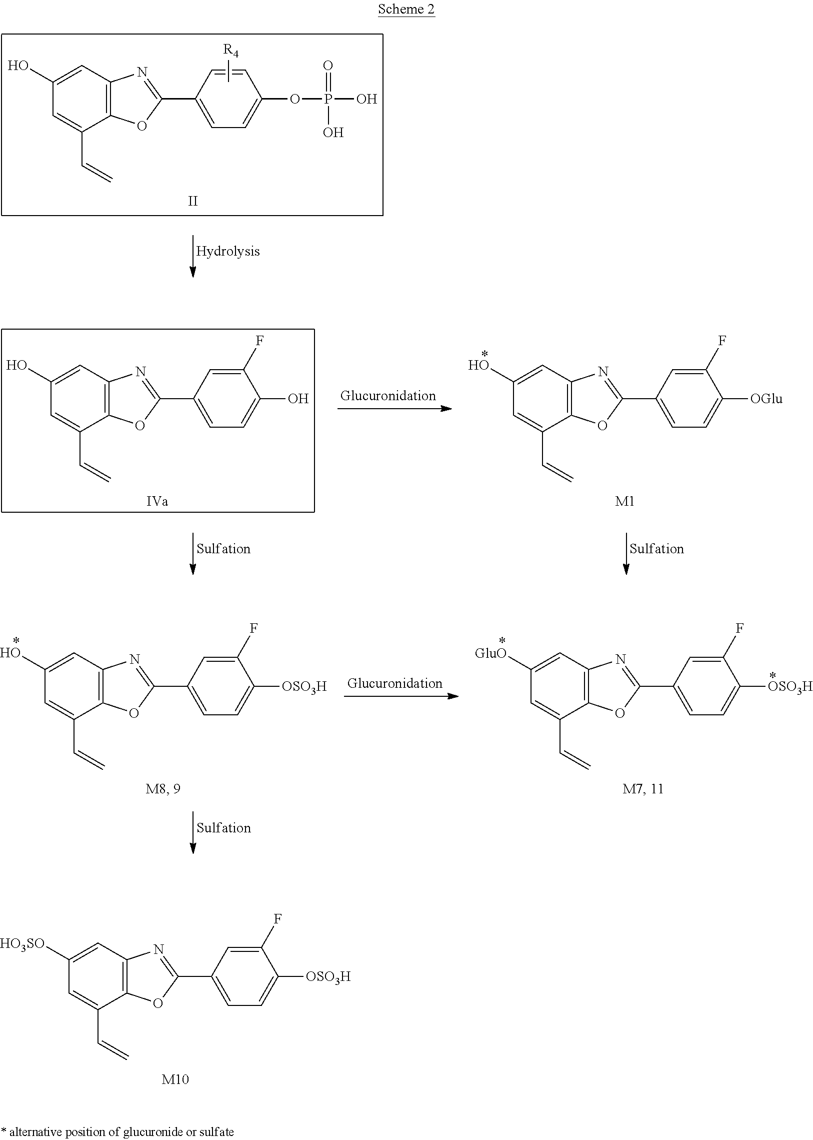

TABLE 2
Metabolites of phosphate prodrug Characterized
in ASD Mouse, Rat and Monkey

| | | | Species | | |
|---|---|---|---|---|---|
| Peak | Metabolite Name | [M − H]⁻ | Mouse | Rat | Monkey |
| M1 | Root compound | 446.0887 | Yes | Yes | Yes |
| | glucuronide | | | | |
| M7 | Root compound | 526.0453 | No | Yes | No |
| | glucuronide sulfate | | | | |
| M8 | Root compound sulfate | 350.0137 | No | Yes | Yes |
| M9 | Root compound sulfate | 350.0134 | No | Yes | Yes |
| M10 | Root compound | 429.9705 | No | Yes | Yes |
| | disulfate | | | | |
| M11 | Root compound | 526.0454 | No | Yes | No |
| | glucuronide sulfate | | | | |
| Root | | 270.0569 | Yes | Yes | Yes |
| compound | | | | | |
| Phosphate | | 350.0229 | Yes | Yes | Yes |
| prodrug | | | | | |

It is intended that each of the patents, applications, and printed publications, including books, mentioned in this patent document be hereby incorporated by reference in their entirety.

As those skilled in the art will appreciate, numerous changes and modifications may be made to the preferred embodiments of the invention without departing from the spirit of the invention. It is intended that all such variations fail within the scope of the invention.

What is claimed is:

1. A compound of Formula I, having the structure: 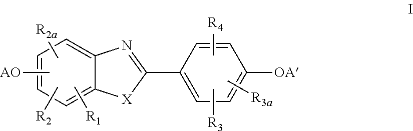
or a pharmaceutically acceptable salt thereof;
wherein:
R₁ is hydrogen, hydroxyl, halogen, alkyl of 1-6 carbon atoms, trifluoroalkyl of 1-6 carbon atoms, cycloalkyl of 3-8 carbon atoms, alkoxy of 1-6 carbon atoms, trifluoroalkoxy of 1-6 carbon atoms, thioalkyl of 1-6 carbon atoms, sulfoxoalkyl of 1-6 carbon atoms, sulfonoalkyl of 1-6 carbon atoms, aryl of 6-10 carbon atoms, a 5 or 6-membered heterocyclic ring having 1 to 4 heteroatoms selected from O, N, S, —NO₂, NR₅R₆, —N(R₅)COR₆, —CN, —CHFCN, CF₂CN, alkynyl of 2-7 carbon atoms, alkenyl of 2-7 carbon atoms; wherein the alkyl or alkenyl moieties are optionally substituted with hydroxyl, —CN, halogen, trifluoroalkyl of 1-6 carbon atoms, trifluoroalkoxy of 1-6 carbon atoms, —COR₅, —CO₂R₅, —NO₂, CONR₅R₆, NR₅R₆, or N(R₅)COR₆;
R₂ and R₂ₐ are each, independently, hydrogen, hydroxyl, halogen, alkyl of 1-6 carbon atoms, alkoxy of 1-4 carbon atoms, alkenyl of 2-7 carbon atoms, alkynyl of 2-7 carbon atoms, trifluoroalkyl of 1-6 carbon atoms, or trifluoroalkoxy of 1-6 carbon atoms; wherein the alkyl, alkenyl, or alkynyl moieties are optionally substituted with hydroxyl, —CN, halogen, trifluoroalkyl of 1-6 carbon atoms, trifluoroalkoxy of 1-6 carbon atoms, —COR₅, —CO₂R₅, —NO₂, CONR₅R₆, NR₅R₆, or N(R₅)COR₆;
R₃ and R₃ₐ are each, independently, hydrogen, alkyl of 1-6 carbon atoms, alkenyl of 2-7 carbon atoms, alkynyl of 2-7 carbon atoms, halogen, alkoxy of 1-4 carbon atoms, trifluoroalkyl of 1-6 carbon atoms, or trifluoroalkoxy of 1-6 carbon atoms; wherein the alkyl, alkenyl, or alkynyl moieties are optionally substituted with hydroxyl, —CN, halogen, trifluoroalkyl of 1-6 carbon atoms, trifluoroalkoxy of 1-6 carbon atoms, —COR₅, —CO₂R₅, —NO₂, CONR₅R₆, NR₅R₆, NR₅R₆ or N(R₅)COR₆;
R₄ is hydrogen, halogen, or alkyl of 1-6 carbon atoms provided that when R₄ is hydrogen, R₁, R₂, R₂ₐ, R₃, and R₃ₐ, cannot all be hydrogen;
R₅ and R₆ are each, independently, hydrogen, alkyl of 1-6 carbon atoms, aryl of 6-10 carbon atoms;
X is O, S, or NR₇;
R₇ is hydrogen, alkyl of 1-6 carbon atoms, aryl of 6-10 carbon atoms, —COR₅, —CO₂R₅ or SO₂R₅;
A and A′ are each independently hydrogen or —P(O)(OR⁸)(OR⁹); wherein at least one of A and A′ is —P(O)(OR⁸)(OR⁹);
R₈ and R₉ are each independently selected from H, C₁₋₁₀ alkyl, C₁₋₁₀ haloalkyl, C₂₋₁₀ alkenyl, C₂₋₁₀ alkynyl, C₆₋₁₀ aryl, C₃₋₈ cycloalkyl, C₅₋₁₀ heteroaryl, C₃₋₁₂ heterocycloalkyl, C₈₋₂₀ arylalkyl, C₈₋₂₀ heteroarylalkyl, C₃₋₂₀ cycloalkylalkyl and C₃₋₂₀ heterocycloalkylalkyl, wherein each of the C₁₋₁₀ alkyl, C₁₋₁₀ haloalkyl, C₂₋₁₀ alkenyl, C₂₋₁₀ alkynyl, C₆₋₁₀ aryl, C₃₋₈ cycloalkyl, C₅₋₁₀ heteroaryl, C₃₋₁₂ heterocycloalkyl, C₆₋₂₀ arylalkyl, C₆₋₂₀ heteroarylalkyl, C₃₋₂₀ cycloalkylalkyl and C₃₋₂₀ heterocycloalkylalkyl, is optionally substituted by 1, 2, 3, 4 or 5 R₁₀;
each R₁₀ is independently halo, C₁₋₆ alkyl, C₁₋₆ haloalkyl, C₆-₁₃ aryl, C₃₋₈ cycloalkyl, C₅₋₁₀ heteroaryl, C₃₋₁₂ heterocycloalkyl, C₆₋₂₀ arylalkyl, C₆₋₂₀ heteroarylalkyl, C₃₋₂₀ cycloalkylalkyl and C₃₋₂₀ heterocycloalkylalkyl, CN, NO₂, OR^{a}, SR^{a}, C(═O)R^{b}, C(═O)NR^{c}R^{d}, C(═O)OR^{a}, OC(═O)R^{b}, OC(═O)NR^{c}R^{d}, NR^{c}R^{d}, NR^{c}C(═O)R^{b}, NR^{c}C(═O)OR^{a}, NR^{c}S(═O)₂R^{b}, S(═O)R^{b}, S(═O)NR^{c}R^{d}, S(═O)₂R^{b}, or S(═O)₂NR^{c}R^{d};
each R^{a} is independently selected from H, C₁₋₆ alkyl, C₁₋₆ haloalkyl, C₂₋₆ alkenyl, C₂₋₆ alkynyl, C₆₋₁₀ aryl, C₃₋₈ cycloalkyl, C₆₋₁₀ heteroaryl, C₃₋₁₂ heterocycloalkyl, C₆₋₂₀ arylalkyl, C₆₋₂₀ heteroarylalkyl, C₃₋₂₀ cycloalkylalkyl and C₃₋₂₀ heterocycloalkylalkyl, wherein each of said C₁₋₆ alkyl, C₁₋₆ haloalkyl, C₂₋₆ alkenyl, C₂₋₆ alkynyl, C₆₋₁₀ aryl, C₃₋₈ cycloalkyl, C₅₋₁₀ heteroaryl, C₃₋₁₂ heterocycloalkyl, C₆₋₂₀ arylalkyl, C₆₋₂₀ heteroarylalkyl, C₃₋₂₀ cycloalkylalkyl and C₃₋₂₀ cycloalkylalkyl and C₃₋₂₀ heterocycloalkylalkyl, is optionally substituted by OH, C₁₋₆ alkoxy, C₁₋₆ haloalkoxy, amino, halo, C₁₋₆ alkyl, C₁₋₆ haloalkyl, C₆₋₁₀ aryl, C₆₋₂₀ arylalkyl, C₅₋₁₀ heteroaryl, C₆₋₂₀ heteroarylalkyl, C₃₋₈ cycloalkyl or C₃₋₁₂ heterocycloalkyl;
each R^{b} is independently selected from H, C₁₋₆ alkyl, C₁₋₆ haloalkyl, C₂₋₆ alkenyl, C₂₋₆ alkynyl, C₆₋₁₀ aryl, C₃₋₈ cycloalkyl, C₆₋₁₀ heteroaryl, C₃₋₁₂ heterocycloalkyl, C₆₋₂₀ arylalkyl, C₆₋₂₀ heteroarylalkyl, C₃₋₂₀ cycloalkylalkyl and C₃₋₂₀ heterocycloalkylalkyl, wherein each of said C₁₋₆ alkyl, C₁₋₆ haloalkyl, C₂₋₅ alkenyl, C₂₋₆ alkynyl, C₆₋₁₀ aryl, C₃₋₈ cycloalkyl, heteroaryl, C₃₋₁₂ heterocycloalkyl, C₆₋₂₀ arylalkyl, C₆₋₂₀ heteroarylalkyl, C₃₋₂₀ cycloalkylalkyl and C₃₋₂₀ heterocycloalkylalkyl, is optionally substituted by OH, C₁₋₆ alkoxy, C₁₋₆ haloalkoxy, amino, halo, C₁₋₆ alkyl, C₁₋₆ haloalkyl, C₆₋₁₀ aryl, C₆₋₂₀ arylalkyl, C-₅₋₁₀ heteroaryl, C₆₋₂₀ heteroalkyl, C₃₋₈ cycloalkyl or C₃₋₁₂ heterocycloalkyl; and
R^{c} and R^{d} are each, independently, selected from H, C₁₋₁₀ alkyl, C₁₋₆, haloalkyl, C₂₋₆ alkenyl, C₂₋₆ alkynyl, C₆₋₁₀ aryl, C₃₋₈ cycloalkyl, C₅₋₁₀ heteroaryl, C₃₋₁₂ heterocycloalkyl, C₆₋₂₀ arylalkyl, C₆₋₂₀ heteroarylalkyl, C₃₋₂₀ cycloalkylalkyl and C₃₋₂₀ heterocycloalkylalkyl, wherein each of said C₁₋₁₀ alkyl, C₁₋₆ haloalkyl, C₂₋₆ alkenyl, C₂₋₆ alkynyl, C₆₋₁₀ aryl, C₃₋₈ cycloalkyl, C₅₋₁₀ heteroaryl, C₃₋₁₂ heterocycloalkyl, C₆₋₂₀ arylalkyl, C₆₋₂₀ heteroarylalkyl, C₃₋₂₀ cycloalkylalkyl and C₃₋₂₀ heterocycloalkylalkyl, is optionally substituted by OH, C₁₋₆ alkoxy, C₁₋₆ haloalkoxy, amino, halo, C₁₋₆ alkyl, C₁₋₆ haloalkyl, C₆₋₁₀ aryl, C₆₋₂₀ arylalkyl, C₅₋₁₀ heteroaryl, C₆₋₂₀ heteroarylalkyl, C₃₋₈ cycloalkyl or C₃₋₁₂ heterocycloalkyl; or
R^{c} and R^{d} together with the N atom to which they are attached form a 4-, 5-, 6- or 7-membered heterocycloalkyl group.

2. The compound of claim 1 wherein R₈ and R₉ are each independently selected from H, C₁₋₁₀ alkyl, C₁₋₁₀ haloalkyl, C₂₋₁₀ alkenyl, C₂₋₁₀ alkynyl, C₆₋₁₀ aryl, C₃₋₈ cycloalkyl C₃₋₁₂ heterocycloalkyl, C₆₋₂₀ arylalkyl, C₆₋₂₀ heteroarylalkyl, C₃₋₂₀ cycloalkylalkyl and C₃₋₂₀ heterocycloalkylalkyl, wherein each of the C₁₋₁₀ alkyl, C₁₋₁₀ haloalkyl, C₂₋₁₀ alkenyl, C₂₋₁₀ alkynyl, C₆₋₁₀ aryl, C₃₋₈ cycloalkyl, C₅₋₁₀ heteroaryl, C₃₋₁₂ heterocycloalkyl, C₆₋₂₀ arylalkyl, C₆₋₂₀ heteroarylalkyl, C₃₋₂₀ cycloalkylalkyl and C₃₋₂₀ heterocycloalkylalkyl, is optionally substituted by 1, 2, 3, 4 or 5 substituents independently selected from halo, C₁₋₄ alkyl, C₁₋₄ haloalkyl, C₆₋₁₀ aryl, C₃₋₈ cycloalkyl, C₅₋₁₀ heteroaryl, C₃₋₁₂ heterocycloalkyl, C₆₋₂₀ arylalkyl, C₆₋₂₀ heteroarylalkyl, C₃₋₂₀ cycloalkylalkyl and C₃₋₂₀ heterocycloalkylalkyl, CN, NO₂, OH, C₁₋₄ alkoxy, C₁₋₄ haloalkoxy, C₆₋₁₀ aryloxy, C₆₋₁₀ heteroaryloxy, C₆₋₂₀ arylalkyloxy, C₆₋₂₀ heteroarylalkyloxy, amino, C₁₋₄ alkylamino, C₂₋₈ dialkylamino, SH, —S—(C₁₋₄ alkyl), C(═O)H, C(═O)—(C₁₋₄ alkyl), C(═O)—(C₆₋₁₀ aryl), C(═O)—(C₆₋₂₀ arylalkyl), C(═O)NH₂, C(═O)NH(C₁₋₄ alkyl), C(═O)N(C₁₋₄ alkyl)₂, C(═O)OH, C(═O)O—(C₁₋₄ alkyl), C(═O)O—(C₆₋₂₀ arylalkyl), OC(═O)H, OC(═O)—(C₁₋₄ alkyl), OC(═O)—(C₆₋₁₀ aryl), OC(═O)—(C₆₋₂₀ arylalkyl), OC(═O)NH₂, OC(═O)NH(C₁₋₄ alkyl), OC(═O)NH—(C₆₋₂₀ arylalkyl), OC(═O)N(C₁₋₄ alkyl)₂, NHC(═O)—(C₁₋₄ alkyl), NHC(═O)—(C₆₋₁₀ aryl), NHC(═O)—(C₆₋₂₀ arylalkyl), N(C₁₋₄ alkyl)C(═O)—(C₁₋₄ alkyl), N(C₁₋₄ alkyl)C(═O)—(C₆₋₁₀ aryl), N(C₁₋₄ alkyl)C(═O)—(C₆₋₂₀ arylalkyl), NHC(═O)O—(C₆₋₂₀ arylalkyl), NHC(═O)O—(C₁₋₄ alkyl), NHC(═O)O—(C₆₋₂₀ arylalkyl), NHC(═O)NH(C₁₋₄ alkyl), NHC(═O)NH—(C₆₋₁₀ aryl), NHC(═O)NH—(C₅₋₂₀ arylalkyl), NHC(═O)NH(C₁₋₄ alkyl)₂, N(C₁₋₄ alkyl)C(═O)NH(C₁₋₄ alkyl), N(C₁₋₄ alkyl)C(═O)NH-(aryl), N(C₁₋₄ alkyl)C(═O)NH—(C₆₋₂₀ arylalkyl), N(C₁₋₄ alkyl)C(═O)NH(C₁₋₄ alkyl)₂, NHS(═O)₂—(C₁₋₄ alkyl), NHS(═O)₂—(C₆₋₁₀ aryl), NHS(═O)₂—(C₆₋₂₀ arylalkyl), S(═O)₂—(C₁₋₄ alkyl), S(═O)₂—(C₆₋₁₀ aryl), S(═O)₂—(C₆₋₂₀ arylalkyl), S(═O)₂NH(C₁₋₄ alkyl), S(═O)₂NH(C₆₋₁₀ aryl), and S(═O)₂NH(C₆₋₂₀ arylalkyl).

3. The compound of claim 1 or 2 wherein R₈ and R₉ are each independently selected from H, C₁₋₁₀ alkyl, C₁₋₁₀ haloalkyl, C₂₋₁₀ alkenyl, C₂₋₁₀ alkynyl, C₆₋₁₀ aryl, C₃₋₈ cycloalkyl, C₅₋₁₀ heteroaryl, C₃₋₁₂ heterocycloalkyl, C₆₋₂₀ arylalkyl, C₆₋₂₀ heteroarylalkyl, C₃₋₂₀ cycloalkylalkyl and C₃₋₂₀ heterocycloalkylalkyl, wherein each of the C₁₋₁₀ alkyl, C₁₋₁₀ haloalkyl, C₂₋₁₀ alkenyl, C₂₋₁₀ alkynyl, C₆₋₁₀ aryl, C₃₋₈ cycloalkyl, C₅₋₁₀ heteroaryl, C₃₋₁₂ heterocycloalkyl, C₆₋₂₀ arylalkyl, C₆₋₂₀ heteroarylalkyl, C₃₋₂₀ cycloalkylalkyl and C₃₋₂₀ heterocycloalkylalkyl₁₃ is optionally substituted by 1, 2 or 3 substituents independently selected from halo, C₁₋₄ alkyl, C₁₋₄ haloalkyl, C₆₋₁₀ aryl, C₃₋₈ cycloalkyl, C₅₋₁₀ heteroaryl, C₃₋₁₂ heterocycloalkyl, C₆₋₂₀ arylalkyl, C₆₋₂₀ heteroarylalkyl, C₃₋₂₀ cycloalkylalkyl and C₃₋₂₀ heterocycloalkylalkyl, CN, NO₂, OH, C₁₋₄ alkoxy, C₁₋₄ haloalkoxy, amino, C₁₋₄ alkylamino and C₂₋₈ dialkylamino.

4. The compound of any one of claims 1 to 3 wherein R₈ and R₉ are each independently selected from H, C₁₋₁₀ alkyl, C₁₋₁₀ haloalkyl, C₂₋₁₀ alkenyl, C₂₋₁₀ alkynyl, C₆₋₁₀ aryl, C₃₋₈ cycloalkyl, C₅₋₁₀ heteroaryl, C₃₋₁₂ heterocycloalkyl, C₆₋₂₀ arylalkyl, C₆₋₂₀ heteroarylalkyl, C₃₋₂₀ cycloalkyl and C₃₋₂₀ heterocycloalkylalkyl.

5. The compound of any one of claims 1 to 4 wherein R₈ and R₉ are each independently selected from H, C₁₋₁₀ alkyl and C₁₋₁₀ haloalkyl.

6. The compound of any one of claims 1 to 5 wherein R₈ and R₉ are each independently selected from H, C₁₋₆ alkyl and C₁₋₆ haloalkyl.

7. The compound of any one of claims 1 to 6 wherein R₈ and R₉ are each independently selected from H and C₁₋₆ alkyl.

8. The compound of any one of claims 1 to 7 wherein R₈ and R₉ are each independently selected from H, and C₁₋₄ alkyl.

9. The compound of any one of claims 1 to 8, wherein:
R₁ is alkenyl of 2-7 carbon atoms; wherein the alkenyl moiety is optionally substituted with hydroxyl, —CN, halogen, trifluoroalkyl of 1-6 carbon atoms, trifluoroalkoxy of 1-6 carbon atoms, —COR₅, —CO₂R₅, —NO₂, CONR₅R₆, NR₅R₆, or N(R₅)COR₆.

10. A compound of any one of claims 1 to 9, wherein the compound has the structure of Formula Ia: 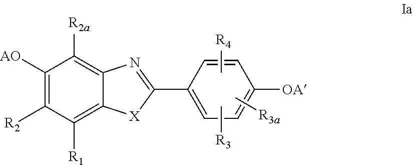
or is a pharmaceutically acceptable salt thereof.

11. The compound of any one of claims 1 to 10, wherein:
A′ is —P(O)(OR⁸)(OR⁹);
X is O;
R₁ is ethylene;
R₂, R₂ₐ, R₃, and R₃ₐ are hydrogen; and
R₄ is halogen.

12. The compound of any one of claims 1 to 11, wherein:
R₄ is fluoro.

13. The compound of any one of claims 1 to 12, wherein the compound has the structure of Formula II: 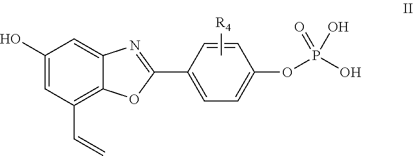
or is a pharmaceutically acceptable salt thereof.

14. A compound of claim 1, selected from:
a) a phosphate derivative of 2-(3-fluoro-4-hydroxyphenyl)-1,3-benzoxazol-5-ol;
b) a phosphate derivative of 2-(3-fluoro-4-hydroxyphenyl)-7-vinyl-1,3-benzoxazol-5-ol;
c) a phosphate derivative of 2-(3-chloro-4-hydroxyphenyl)-1,3-benzoxazol-5-ol;
d) a phosphate derivative of 2-(2-chloro-4-hydroxyphenyl)-1,3-benzoxazol-5-ol;
e) a phosphate derivative of 2-(3-fluoro-4-hydroxyphenyl)-1,3-benzoxazol-6-ol;
f) a phosphate derivative of 2-(3-tent-butyl-4-hydroxyphenyl)-1,3-benzoxazol-6-ol;
g) a phosphate derivative of 2-(3-chloro-4-hydroxyphenyl)-1,3-benzoxazol-6-ol;
h) a phosphate derivative of 6-chloro-2-(3-fluoro-4-hydroxyphenyl)-1,3-benzoxazol-5-ol;
i) a phosphate derivative of 6-bromo-2-(3-fluoro-4-hydroxyphenyl)-1,3-benzoxazol-5-ol;
j) a phosphate derivative of 6-chloro-2-(4-hydroxyphenyl)-1,3-benzoxazol-5-ol;
k) a phosphate derivative of 5-chloro-2-(4-hydroxyphenyl)-1,3-benzoxazol-6-ol;
l) a phosphate derivative of 7-bromo-2-(3-fluoro-4-hydroxyphenyl)-1,3-benzoxazol-5-ol;
m) a phosphate derivative of 7-bromo-2-(2-fluoro-4-hydroxyphenyl)-1,3-benzoxazol-5-ol;
n) a phosphate derivative of 7-bromo-2-(2,3-difluoro-4-hydroxyphenyl)-1,3-benzoxazol-5-ol;
o) a phosphate derivative of 2-(4-hydroxyphenyl)-7-vinyl-1,3-benzoxazol-5-ol;
p) a phosphate derivative of 7-(1,2-dibromoethyl)-2-(4-hydroxyphenyl)-1,3-benzoxazol-5-ol;
q) a phosphate derivative of 7-(1-bromovinyl)-2-(4-hydroxyphenyl)-1,3-benzoxazol-5-ol;
r) a phosphate derivative of 7-ethynyl-2-(4-hydroxyphenyl)-1,3-benzoxazol-5-ol;
s) a phosphate derivative of 2-(4-hydroxyphenyl)-7-propyl-1,3-benzoxazol-5-ol;
t) a phosphate derivative of 7-butyl-2-(4-hydroxyphenyl)-1,3-benzoxazol-5-ol;
u) a phosphate derivative of 7-cyclopentyl-2-(4-hydroxyphenyl)-1,3-benzoxazol-5-ol;
v) a phosphate derivative of ethyl 5-hydroxy-2-(4-hydroxyphenyl)-1,3-benzoxazole-7-carboxylate;
w) a phosphate derivative of 2-(4-hydroxyphenyl)-7-phenyl-1,3-benzoxazol-5-ol;
x) a phosphate derivative of 2-(4-hydroxyphenyl)-7-methoxy-1,3-benzoxazol-5-ol;
y) a phosphate derivative of 7-ethyl-2-(4-hydroxyphenyl)-1,3-benzoxazol-5-ol;
z) a phosphate derivative of 7-ethyl-2-(2-ethyl-4-hydroxyphenyl)-1,3-benzoxazol-5-ol;
aa) a phosphate derivative of 5-hydroxy-2-(4-hydroxyphenyl)-1,3-benzoxazole-7-carbaldehyde;
bb) a phosphate derivative of 7-(hydroxymethyl)-2-(4-hydroxyphenyl)-1,3-benzoxazol-5-ol;
cc) a phosphate derivative of 7-(bromomethyl)-2-(4-hydroxyphenyl)-1,3-benzoxazol-5-ol;
dd) a phosphate derivative of [5-hydroxy-2-(4-hydroxyphenyl)-1,3-benzoxazol-7-yl]acetonitrile;
ee) a phosphate derivative of 7-(1-hydroxy-1-methylethyl)-2-(4-hydroxyphenyl)-1,3-benzoxazol-5-ol;
ff) a phosphate derivative of 2-(4-hydroxyphenyl)-7-isopropenyl-1,3-benzoxazol-5-ol;
gg) a phosphate derivative of 2-(4-hydroxyphenyl)-7-isopropyl-1,3-benzoxazol-5-ol;
hh) a phosphate derivative of 7-bromo-2-(4-hydroxy-3-(trifluoromethyl)phenyl)-1,3-benzoxazol-5-ol;
ii) a phosphate derivative of 7-(2-furyl)-2-(4-hydroxyphenyl)-1,3-benzoxazol-5-ol;
jj) a phosphate derivative of 2-(3-fluoro-4-hydroxyphenyl)-7-(2-furyl)-1,3-benzoxazol-5-ol;
kk) a phosphate derivative of 2-(4-hydroxyphenyl)-7-thien-2-yl-1,3-benzoxazol-5-ol;
ll) a phosphate derivative of 2-(4-hydroxyphenyl)-7-(1,3-thiazol-2-yl)-1,3-benzoxazol-5-ol;
mm) a phosphate derivative of 2-(3-fluoro-4-hydroxyphenyl)-5-hydroxy-1,3-benzoxazole-7-carbonitrile;
nn) a phosphate derivative of 4-bromo-2-(4-hydroxyphenyl)-7-methoxy-1,3-benzoxazol-5-ol;
oo) a phosphate derivative of 4,6-dibromo-2-(4-hydroxyphenyl)-7-methoxy-1,3-benzoxazol-5-ol; and
pp) a phosphate derivative of 7-bromo-2-(3,5-difluoro-4-hydroxyphenyl)-1,3-benzoxazol-5-ol;
or a pharmaceutically acceptable salt thereof.

15. A compound according to claim 1 that is selected from 2-fluoro-4-(5-hydroxy-7-vinylbenzo[d]oxazol-2-yl)phenyl dihydrogen phosphate; 2-(3-fluoro-4-hydroxyphenyl)-7-vinylbenzo[d]oxazol-5-yl dihydrogen phosphate; and 2-fluoro-4-(5-(di-hydroxy)phosphoryloxy)-7-vinylbenzo[d]oxazol-2-ylphenyl dihydrogen phosphate; or a pharmaceutically acceptable salt thereof.

16. A compound according to claim 1 selected from the group consisting of a compound of Formula V: 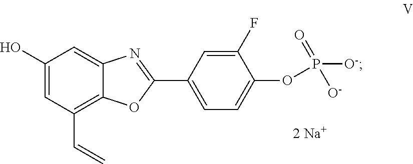
a compound of Formula VI: 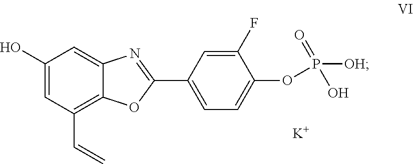
and a compound of Formula VII:

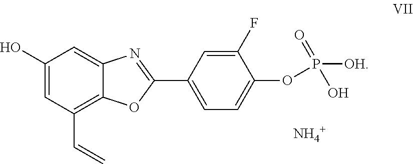

17. A composition comprising the compound of any one of claims 1 to 16, and a pharmaceutically acceptable carrier.

18. The composition of claim 17 wherein said pharmaceutically acceptable carrier is an aqueous solvent.

19. A method for treating or inhibiting a disease, disorder, or condition in a mammal, comprising the steps of:
a) identifying a mammal having said disease, disorder, or condition; and
b) administering to said mammal a therapeutically effective amount of a compound of any one of claims 1 to 16; and
wherein said disease, disorder, or condition is selected from: an inflammatory disease, disorder, or condition; cancer; a cardiovascular disease, disorder, or condition; a cognitive disease, disorder, or condition; a disease, disorder, or condition of the skin; a neurodegenerative disease, disorder, or condition; diabetes; a disease, disorder, or condition associated with peri-menopause, menopause, or post-menopause; and a disease, disorder or condition associated with a dysregulated systemic inflammatory response.

20. The method of claim 19, wherein said therapeutically effective amount of the compound is administered parenterally.

21. The method of claim 19 or 20, wherein said disease, disorder, or condition is an inflammatory disease, disorder, or condition selected from the group consisting of: prostatitis; interstitial cystitis; inflammatory bowel disease; Crohn's disease; ulcerative proctitis; colitis; arthritis; joint swelling or erosion; prostatic hypertrophy; asthma; pleurisy; and joint damage secondary to arthroscopic or surgical procedures; a cancer selected from the group consisting of: uterine leiomyomas, breast cancer; endometrial cancer; polycystic ovary syndrome; endometrial polyps; benign breast disease; adenomyosis; ovarian cancer; melanoma; prostrate cancer; colon cancer; glioma; and astioblastomia; a cardiovascular disease, disorder, or condition selected from the group consisting of: aberrant cholesterol, triglyceride, Lp(a), or LDL levels; hypercholesteremia; hyperlipidemia; atherosclerosis; hypertension; peripheral vascular disease; restenosis; vasospasm; and vascular wall damage from cellular events leading toward immune mediated vascular damage; a cognitive disease, disorder, or condition selected from the group consisting of: senile dementia; Alzheimer's disease; cognitive decline; stroke; anxiety; and free radical induced disease states; a disease, disorder, or condition of the skin selected from the group consisting of: psoriasis and dermatitis; a neurodegenerative disease, disorder, or condition selected from the group consisting of: ischemia; reperfusion injury; multiple sclerosis; systemic lupus erythematosis; uveitis; and hemmorhagic shock; a disease, disorder, or condition associated with peri-menopause, menopause, or post-menopause selected from the group consisting of: vaginal or vulvar atrophy; atrophic vaginitis; vaginal dryness; pruritus; dyspareunia; dysuria; frequent urination; urinary incontinence; urinary tract infections; vasomotor symptoms; endometriosis; and conception; a disease, disorder or condition associated with a dysregulated systemic inflammatory response selected from the group consisting of: sepsis; multiple organ failure; and septic shock.

22. The method of claim 21, wherein said arthritis is rheumatoid arthritis.

23. A process for preparing a compound of formula I according to any one of claims 1 to 16, comprising the step of:
phosphorylating a compound of Formula IV: 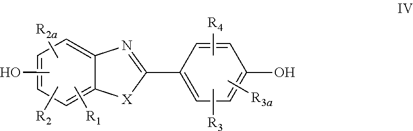
or a salt thereof with a phosphorylating reagent;
wherein:
R₁, R₂, R₂ₐ, R₃, R₃ₐ, R₄, and X are as defined in claims 1 to 16.

24. The process of claim 23, wherein said compound has the structure of Formula III:

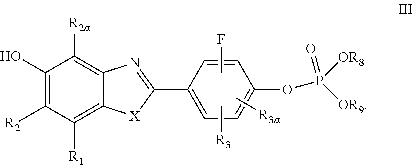

25. The process of claim 24, wherein said compound of Formula III has a structure of Formula IIIa:

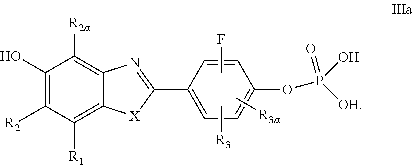

26. The process of any one of claims 23 to 25 wherein the phosphorylating agent comprises diethyl phosphate.

27. The process of any one of claims 23 to 25, wherein the phosphorylating agent comprises diethyl chlorophosphate.

28. The process of any one of claims 23 to 27, wherein the phosphorylating reaction is carried out in the solvent system in the presence of a base.

29. The process of any one claims 23 to 28, further comprising the step of isolating a salt of a compound of Formula I, wherein the salt has the Formula Ib:
[R₁₁—O—PO₃⁻²]M
Ib
wherein:
R₁₁ is 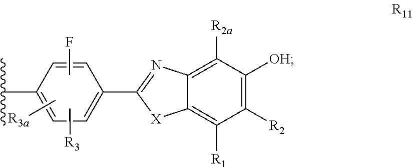
and
M is a Group I or II metal ion.
